(19) United States
(12) Patent Application Publication
(10) Pub. No.: US 2002/0012982 A1
(43) Pub. Date: Jan. 31, 2002

(54) Methods and compositions for rapid protein and peptide extraction and isolation using a lysis matrix

(75) Inventors: Robert W. Blakesley, Frederick, MD (US); Barbara Flynn, Ellicott City, MD (US); Peter Clausen, Ijamsville, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Invitrogen Corporation

(21) Appl. No.: 09/903,864

(22) Filed: Jul. 13, 2001

Related U.S. Application Data
(60) Provisional application No. 60/274,630, filed on Mar. 12, 2001.
(60) Provisional application No. 60/218,081, filed on Jul. 13, 2000.

(51) Int. Cl. C12N 9/00; C07K 1/14

(52) U.S. Cl. 435/183; 530/412

(57) ABSTRACT

The present invention relates generally to compositions, methods and kits for use in extracting and isolating protein or peptide molecules. More specifically, the invention relates to such compositions, methods and kits that are useful in the isolation of protein or peptide molecules from cells (e.g., bacterial cells, animal cells, fungal cells, viruses, yeast cells or plant cells) via lysis and one or more additional isolation procedures, such as one or more filtration and/or chromatography procedures. In particular, the invention relates to compositions, methods and kits wherein protein or peptide molecules are isolated using an integrated lysis/filtration matrix, which may comprise one or more supports (e.g., polyolefin, scintered polyethylene, nitrocellulose, polypropylene, polycarbonate, cellulose acetate, silica, and the like). The compositions, methods and kits of the invention are suitable for isolating a variety of forms of protein or peptide molecules from cells. The compositions, methods and kits of the invention are particularly well-suited for rapid isolation of recombinant protein or peptide molecules expressed in bacterial cells, either as soluble protein, or as an inclusion body. The invention is particularly useful in high throughput applications, allowing quick isolation and/or analysis of proteins and/or peptides from numerous sources.

### BACKGROUND OF THE INVENTION

1. Field of the Invention

The present invention is in the fields of molecular biology and protein biochemistry. The invention relates generally to compositions, methods and kits for use in extracting and isolating protein and peptide molecules. More specifically, the invention relates to such compositions, methods and kits that are useful in the isolation of protein and peptide molecules from cells via lysis and one or more additional isolation procedures, such as one or more chromatography/filtration separations. The compositions, methods and kits of the invention are suitable for isolating a variety of forms of protein and peptide molecules from cells.

2. Background Art

The first step in the purification of native and recombinant proteins is the lysis of the cells producing said proteins, resulting in liberation of the cellular components. Classic physical methods for cell lysis include sonication and the use of a French Pressure Cell, often in combination with a chemical or enzyme agent to aid in lysis. Lysis by physical methods produces membrane fragments and small DNA molecules caused by shearing of the chromosomal DNA, either of which can interfere with subsequent analysis of the desired proteins. Removal of these contaminants requires additional costly and time consuming purification steps.

Several commercial kits are available for the rapid extraction of proteins from cells. Two of the most popular are BugBuster™ (Novagen) and B-PER (Pierce). Both of these kits employ the use of a detergent solution to disrupt the cell membrane, thereby releasing the cellular components including protein. Neither of these methods couple a purification step with the extraction method. The BugBuster™ method utilizes a Benzonase® nuclease to decrease the viscosity in the lysate due to the large amounts of chromosomal DNA present in the sample after lysis. However, the product does not include any method for removal of the small DNA fragments which are necessarily generated by the nuclease digestion. The B-PER product is solely intended as an extraction system. The system includes a centrifugation step, which removes some insoluble debris; however, there is no subsequent purification. Any contamination of the lysates generated with the B-PER product must be removed using separate methods of purification.

Classic protein purification methods include precipitation (e.g. PEI, PEG, and ammonium sulfate), filtration, preparative electrophoresis and the like. These methods are often performed on bacterial lysates or partially purified preparations of protein. Additional methods based on chromatography include, but are not limited to, ion-exchange chromatography, size-exclusion chromatography, hydrophobic interaction chromatography, and affinity chromatography. Any and all of these methods are dependent on an efficient lysis procedure in order to insure adequate yield.

While methods exist in the art for lysis of cells, there exists a need in the art for a rapid method which combines gentle cell lysis; separation of the protein and peptide of interest from contaminating DNA, membrane fragments and cellular debris; and additional purification methods into one or a few procedures. The present invention provides such compositions, methods and kits.

### BRIEF SUMMARY OF THE INVENTION

The present invention relates generally to compositions, methods and kits for use in extracting and isolating protein and peptide molecules. More specifically, the invention relates to such compositions, methods and kits that are useful in the extraction and isolation of protein and peptide molecules from cells (e.g., bacterial cells, animal cells, fungal cells, yeast cells or plant cells) via lysis and one or more additional isolation procedures, such as one or more filtration procedures. In particular, the invention relates to compositions, methods and kits wherein desired protein and peptide molecules are extracted and isolated in one or a few procedures using a lysis/filter matrix.

More particularly, the invention relates to methods for extracting and isolating protein and peptide molecules comprising:

(a) contacting one or more cells or cellular sources with at least one pore-containing matrix which substantially retards the flow of high molecular weight molecules, structures, and aggregates but does not substantially retard the flow of soluble protein and peptide molecules;

(b) causing the one or more cells or cellular source to lyse or disrupt (e.g., disrupt the integrity of the cell membrane and/or cell wall) such that protein or peptide molecules are released from the one or more cells or cellular source; and

(c) collecting the protein and peptide molecules.

In another embodiment, the invention relates to methods for extracting and isolating protein and peptide molecules comprising:

(a) causing the one or more cells or cellular source to lyse or disrupt (e.g., disrupt the integrity of the cell membrane and/or cell wall) such that protein or peptide molecules are released from the one or more cells or cellular source;

(b) contacting one or more cells or cellular sources with at least one pore-containing matrix which substantially retards the flow of high molecular weight molecules, structures, and aggregates but does not substantially retard the flow of soluble protein and peptide molecules; and

(c) collecting the protein and peptide molecules.

In yet another preferred embodiment, the invention relates to methods for extracting and isolating protein and peptide molecules comprising:

(a) contacting the one or more cells or cellular sources with at least one pore-containing matrix which substantially retards the flow of high molecular weight molecules, structures, and aggregates but does not substantially retard the flow of soluble protein and peptide molecules;

(b) causing the one or more cells or cellular source to lyse or disrupt (e.g., disrupt the integrity of the cell membrane and/or cell wall) such that protein or peptide molecules are released from the one or more cells or cellular source;

(c) contacting the filter with an elution/disruption composition which will disrupt and/or solubilize protein aggregates and inclusion bodies; and

(d) collecting said protein or peptide molecules.

In yet another preferred embodiment, the invention relates to methods for extracting and isolating protein and peptide molecules comprising:

(a) causing the one or more cells or cellular source to lyse or disrupt (e.g., disrupt the integrity of the cell membrane and/or cell wall) such that protein or peptide molecules are released from the one or more cells or cellular source;

(b) contacting the one or more cells or cellular sources with at least one pore-containing matrix which substantially retards the flow of high molecular weight molecules, structures, and aggregates but does not substantially retard the flow of soluble protein and peptide molecules;

(c) contacting the filter with an elution/disruption composition which will disrupt and/or solubilize protein aggregates and inclusion bodies; and

(d) collecting said protein or peptide molecules.

In accordance with the invention, the cells may be lysed or disrupted before contacting the cells with the matrix, although cell lysis or disruption preferably takes place after the cells are contacted with the matrix and more preferably at the same time or approximately the same time (e.g., simultaneously or substantially simultaneously) the cells are contacted with the matrix. In another aspect, the cells are preferably trapped within or on the matrix prior to or during cell lysis or disruption. In yet another aspect, the cells are lysed/disrupted by contacting them with a composition or compound which causes or aids in cell lysis or disruption, although mechanical or physical forces (e.g., pressure, sonication, temperature (heating, freezing), and/or freeze-thawing etc.) may be used in accordance with the invention. Any combination of mechanical forces, physical forces or lysis compositions/compounds may be used to disrupt/lyse the cells. Preferably, if soluble protein in its native conformation is desired for functional or structural analysis, cells are lysed or disrupted with an agent that does not substantially perturb the native conformation or function of the desired protein or peptide.

After the one or more cells are lysed/disrupted/permeabilized in accordance with the invention, soluble protein and peptide molecules are substantially separated from larger molecular weight molecules, structures and aggregates. Such separation is preferably accomplished by the matrix retarding the flow of the high molecular weight molecules, structures and aggregates, and not substantially retarding the flow of low molecular weight molecules. For example, chromosomal DNA is considered to be substantially trapped/bound by the matrix if little or no high molecular weight band(s) is observed when analyzing a sample of the eluate by gel electrophoresis (e.g. agarose stained with ethidium bromide). Such binding/trapping action allows physical separation of such molecules where the smaller molecules of interest (e.g. soluble proteins and peptides) are allowed to substantially pass through the matrix while the larger molecules (e.g. chromosomal DNA, membrane fragments, and inclusion bodies) are trapped or bound to the matrix.

In another preferred aspect of the invention, after the one or more cells are lysed/disrupted/permeabilized in accordance with the invention, the soluble proteins and peptides are allowed to pass freely through the filter of the invention, while protein and peptide aggregates and inclusion bodies are retained on/in the filter of the invention. The filter is then contacted with a elution composition (e.g. 6M Urea) that will disrupt the protein or peptide aggregates or inclusion bodies and allow the constituent proteins to flow freely through the filter of the invention.

According to the invention, the matrix may be any porous material that retards the flow of high molecular weight molecules, structures and aggregates, and/or does not substantially retard the flow of low molecular weight molecules. Such matrices may include but are not limited to a polyester matrix, a polyolefin matrix, a scintered polyethylene matrix, a nitrocellulose matrix, a cellulose acetate matrix, a cellulose matrix, a porous ceramic matrix, a silica matrix, a polysaccharide matrix (SEPHAROSE, agarose, SEPHADEX, etc.), a polymer matrix (SEPHACRYL, TRISACRYL, TOYOPEARL, BIO-GEL, etc.) and the like. In a preferred aspect, the matrix is a solid matrix, although the matrix may be a semi-solid matrix. Suitable matrix materials may be obtained commercially, for example from Filtrona Richmond, Inc. (Richmond, Va.), Bio-Rad (Richmond, Calif.), Gentra Systems (Minneapolis, Minn.), Tosohaas (Montgomeryville, Pa.), BioSepra, Inc., (Marlborough, Mass.), and Porex Technologies Corp. (Fairburn, Ga.). In a related aspect, the matrix may be prepared in various sizes, shapes, and forms including flat, wafer, cylindrical, rectangular, beads, gels, square, cartridge, swab tip, plug, frit, membrane and the like, and may also be contained in various containers such as tubes, bottles, vials, ampules, microspin tubes, wells, multi-well plates, bags and the like. In a preferred aspect, the invention involves the use of size separation chromatography and/or filtration to separate or substantially separate soluble protein and peptide molecules from high molecular weight molecules, structures and aggregates. Thus, any matrix which provides desired size separation (e.g., filters, chromatography supports, etc.) may be used in the invention. One of skill in the art can readily determine the appropriate matrix, pore size of the matrix, size, shape and dimensions of the matrix taking into consideration the type and size of the desired protein and peptide molecules and the cell type or cellular source. In another aspect, the invention combines such size separation/filtration with cell lysis/disruption (preferably such lysis/disruption is done when or approximately when the cellular source comes in contact with or after the cellular source is in contact with the filtration matrix). The pores or passage ways in the matrix are typically small enough to prevent passage of large molecules, structures and aggregates, but large enough to permit passage of soluble protein and peptide molecules of interest. The potential pore sizes may range from about 0.1 to about 10,000 microns in diameter, about 0.1 to about 5,000 microns in diameter, about 0.1 to about 1,000 microns in diameter, about 1 to about 500 microns in diameter, about 10 to about 500 microns in diameter, or about 25 to about 400 microns in diameter.

In a preferred embodiment, in addition to the pore-containing matrix, additional multiple matrixes (e.g. one, two, three or more) may be used in the practice of the invention. In one embodiment, an additional pore containing matrix is a porous filter underneath the lysis matrix that filters out any residual cell debris. Such porous filters include glass filter membranes (GF/F), cellulose acetate, polypropylene, polytetrafluoroethylene, polyvinylidiene fluoride, polyethylene and polyethersulfone. Such porous membranes are commercially available, for example, from Whatman, 3M, Gelman and Millipore. The pore sizes may range from about 0.1-10 microns, more preferably, about 0.5-1.5 microns, most preferably, about 0.7-1 micron. A preferred filter is the Whatman GF/F glass fiber filter that has a pore size of 0.7 micron. A further matrix that may be employed is a frit disposed below the other matrix(es) that provides mechanical support, if necessary.

In another preferred embodiment, the composition or compound that disrupts the cellular membrane or cell wall integrity may comprise one or more non-ionic detergents, including, but not limited to, N-octyl-β-D-glucopyranside, N-octyl-β-D-maltoside, ZWITTERGENT 3.14, deoxycholate; n-Dodecanoylsucrose; n-Dodecyl-β-D-glucopyranoside; n-Dodecyl-β-D-maltoside; n-Octyl-β-D-glucopyranoside; n-Octyl-β-D-maltopyranoside; n-Octyl-β-D-thioglucopyranoside; n-Decanoylsucrose; n-Decyl-β-D-maltopyranoside; n-Decyl-β-D-thiomaltoside; n-Heptyl-β-D-glucopyranoside; n-Heptyl-β-D-thioglucopyranoside; n-Hexyl-β-D-glucopyranoside; n-Nonyl-β-D-glucopyranoside; n-Octanoylsucrose; n-Octyl-β-D-glucopyranoside; n-Undecyl-β-D-maltoside; APO-10; APO-12; Big CHAP; Big CHAP, Deoxy; BRIJ® 35; C₁₂E₅; C₁₂E₆; C₁₂E₈; C₁₂E₉; Cyclohexyl-n-ethyl-β-D-maltoside; Cyclohexyl-n-hexyl-β-D-maltoside; Cyclohexyl-n-methyl-β-D-maltoside; Digitonin; ELUGENT™; GENAPOL® C-100; GENAPOL® X-080; GENAPOL® X-100; HECAMEG; MEGA-10; MEGA-8; MEGA-9; NOGA; NP-40; PLURONIC® F-127; TRITON® X-100; TRITON® X-114; TWEEN® 20; or TWEEN® 80. Additionally, an ionic detergent can be used with the methods of the invention, including, but not limited to BATC, Cetyltrimethylammonium Bromide, Chenodeoxycholic Acid, Cholic Acid, Deoxycholic Acid, Glycocholic Acid, Glycodeoxycholic Acid, Glycolithocholic Acid, Lauroylsarcosine, Taurochenodeoxycholic Acid, Taurocholic Acid, Taurodehydrocholic Acid, Taurolithocholic Acid, Tauroursodeoxycholic Acid, and TOPPA. Zwitterionic detergents can also be used with the compositions and methods of the invention, including, but not limited to, amidosulfobetaines, CHAPS, CHAPSO, carboxybetaines, and methylbetaines. In addition one or more enzymes such as zymolyase, lyticase, lysozyme or lysostaphin; one or more inorganic salts such as sodium chloride, potassium chloride, or lithium chloride; one or more acids and/or bases or buffering agents (e.g., to increase or reduce pH); or any other compound or enzyme which may assist in the disruption of the integrity of (i.e., lyses or causes the formation of pores in) the cell membrane and/or cell walls (e.g., polymixin B) can be used. In another aspect, the composition may comprise one or more compounds or enzymes to degrade, destroy or remove unwanted components or contaminants (e.g., ribonucleases (RNases), DNases, and nucleases (e.g. endonucleases and exonucleases) to remove or destroy or degrade undesired nucleic acid molecules (e.g., DNA or RNA) released from the cellular source). If soluble protein in its native conformation is desired then a non-denaturing detergent should be used. In one particularly preferred aspect, the cell lysis/disruption composition may be adsorbed onto or complexed with or associated with the matrix prior to applying the one or more cells or cellular source to the matrix. In a preferred aspect, the composition is dried in or on the matrix. Thus, in a preferred aspect, the matrix comprises a cell lysis/disruption compound or composition. In this aspect, the cell disruption/lysis may occur when or about the same time the cells come into contact with the composition containing matrix. In another aspect, the composition is added after the cells are added to (e.g., bound to or associated with) the matrix. In yet another aspect, the composition is added to the cells prior to adding the cells to the matrix. In this aspect, the composition may be formulated to weaken the cell membrane/cell wall such that the cells will substantially disrupt/lyse when contacted with the matrix. Alternatively, the composition will substantially lyse/disrupt the cells before addition to the matrix.

In accordance with the invention, the protein and peptide molecules of interest may be removed from the matrix by elution with an aqueous solution, such as a buffered salt solution or elution buffer. The insoluble molecules (e.g. chromosomal or genomic DNA, membrane fragments, protein aggregates and inclusion bodies) are substantially retained in or on the matrix, thus allowing the soluble protein and peptide molecules to be eluted or to be substantially removed from the matrix. Such elution or removal of the soluble protein and peptide molecules, with or without the addition of an aqueous solution, may be facilitated by centrifugation, gravity, vacuum, pressure, etc., which provides flow of the desired protein or peptide sample from the matrix. The soluble protein and peptide molecules of interest may then be further purified by standard protein purification techniques.

In another preferred embodiment of the invention, after the soluble protein and peptide molecules have been eluted or removed from the matrix, the matrix, containing the insoluble materials (e.g. membrane fragments and/or inclusion bodies) is contacted with a second elution/disruption reagent (e.g. 6M Urea) which causes the disruption of the insoluble materials (membrane fragments and/or inclusion bodies), and the solubilization of the constituent proteins. These liberated protein or peptide molecules can then be eluted or substantially removed from the matrix. Such elution or removal of the soluble protein and peptide molecules, with or without the addition of an aqueous solution, may be facilitated by centrifugation, gravity, vacuum, pressure, etc., which provides flow of the desired protein or peptide sample from the matrix. Appropriate compositions included in the second elution buffer include compositions capable of disrupting and solubilizing the protein or peptide molecules present in an inclusion body or membrane fragment as appropriate. Appropriate compositions include, but are not limited to, urea, guanadinium chloride, detergents, chaeotropic agents, salts, and the like. In another aspect of the invention, cell lysis/disruption or disruption/solubilization of insoluble material can be accomplished in one step, preferably with one composition or reagent that serves both functions. Such compositions may comprise, but are not limited to, urea, guanadinium chloride, ionic or non-ionic detergents, and the like.

The methods according to the invention are suitable for isolation of protein and peptide molecules from any cell or cellular source, including bacterial cells (particularly Escherichia coli cells), yeast cells, fungal cells, animal cells (particularly insect cells, and mammalian cells including human cells, CHO cells, VERO cells, Bowes melanoma cells, HepG2 cells, and the like), and plant cells, any of which may be transformed cells, established cell lines, cancer cells, primary cells or normal cells. The methods of the invention are particularly well-suited for isolation of soluble proteins and peptides, including but not limited to proteins and peptides expressed from a cDNA expression library, or recombinant proteins and peptides expressed from plasmids in a prokaryotic or eukaryotic host.

The invention also relates to the isolated protein and peptide molecules produced by the methods of the invention. The invention also relates to further manipulation of the isolated protein and peptide molecules of the invention by standard biochemical or chromatographic techniques such as affinity chromatography, ion-exchange chromatography, hydrophobic interaction chromatography, precipitation and the like.

The invention further relates to immobilizing the protein or peptide molecules of the invention on a solid substrate for the purpose of high throughput screening. Examples of such solid substrates include, but are not limited to, multi-well plates, chips, slides, wafers, filters, sheets, tubes, and the like. Proteins or peptides immobilized on appropriate substrates can then be screened by any method known in the art, including but not limited to, hybridization with an antibody, contacting with a substrate, contacting with a ligand, contacting with a biological macromolecule (e.g. DNA, RNA, protein, peptide, carbohydrate, lipid, amino acid, nucleotide, nucleoside, etc.) and the like. The proteins or peptides immobilized on the substrate can be analyzed for the presence of an appropriate signal, which may include, but is not limited to, fluorescence, chemiluminescence, bioluminescence, absorption of a particular wavelength of light, binding of a particular substrate, changes in color, or any other method deemed appropriate to gain the information desired. The invention also relates to the further characterization or utilization of the isolated proteins or peptides of the invention.

In a related aspect, the invention relates to compositions for use in isolating protein and peptide molecules and to compositions made according to the practice of the invention. Such compositions of the invention preferably comprise one or more components, such as:

(a) one or more cellular sources of the protein or peptide molecules of interest;

(b) at least one pore-containing matrix which substantially retards the flow of high molecular weight molecules, structures or aggregates, but not substantially retard the flow of soluble protein or peptide molecules; and

(c) a cell disrupting or cell lysis portion comprising at least one compound that disrupts the integrity of the cellular membrane or cell wall when the cellular source comes into contact with said compound.

Optionally, the compositions of the invention further include a solubilization reagent capable of solubilizing insoluble material specifically, membrane fragments and/or inclusion bodies.

Preferred cellular sources, solid matrices, and lysis/disrupting/permeabilization compounds for use in the compositions of the invention include those described and used in the methods of the present invention. In a preferred composition of the invention, an effective amount of the compound that disrupts the integrity of the cellular membrane and/or cell wall is adsorbed onto or complexed with or associated with the matrix, for example by ionic, hydrophobic, or covalent or non-covalent attachment of the cell membrane/cell wall disrupting compound to the matrix material. In one embodiment, such compound is dried in or on the matrix. While some commercially available matrixes have small amounts of TRITON detergent incorporated into the fibers for manufacturing purposes, the detergent is not present in an amount large enough to cause substantial lysis/disruption/permeabilization of cells. Thus, according to the present invention, at least one additional cell lysis/disruption/permeabilization composition typically is added or used according to the methods of the invention. The compositions of the invention are useful in isolating a variety of proteins and peptide molecules, particularly those described herein.

The invention also relates to kits for use in isolating protein and peptide molecules, comprising one or more of the components for carrying out the methods of the invention or one or more of the compositions of the invention. Such kits of the invention may comprise one or more components, which may be contained in one or more containers such as boxes, cartons, tubes, vials, ampules, bags, and the like. In one such aspect, the kits of the invention may comprise at least one pore-containing matrix which substantially retards the flow of high molecular weight molecules, structures and aggregates, but does not substantially retard the flow of soluble protein and peptide molecules (and which preferably traps a cellular source protein or peptide within or on the matrix).

Such kits may comprise additional reagents selected from the group consisting of, a cell lysing/disrupting/permeabilizing composition comprising at least one compound that disrupts the integrity of the cellular membrane or cell wall when the cellular source comes into contact with the compound or composition, such that the protein and peptide molecules are released from the cellular source; and a solubilization reagent, capable of solubilizing insoluble material, including, but not limited to, membrane fragments and inclusion bodies.

The at least one pore-containing matrix and cell lysing/disrupting/permeabilizing composition may be provided within a single container.

In one such kit, the matrix comprises the cell lysing/disrupting/permeabilizing composition or compound. An effective amount of such cell lysing/disrupting/permeabilizing composition or compound may be adsorbed onto, complexed with or associated with the matrix, for example by ionic, hydrophobic, non-covalent or covalent attachment to the matrix material. Such cell lysing/disrupting/permeabilizing composition may or may not be dried in or on the matrix. Preferred solid matrix materials, cell lysing/disrupting/permeabilizing compositions and compounds, and washing and elution compositions for use in the kits of the invention include those described herein for use in the methods of the present invention. The kits of the invention further comprise one or more additional reagents, such as one or more components or reagents that may be useful in conjunction with further purification, processing and analysis of the isolated protein and peptide molecules of the invention, for example chromatography resins. Additionally, said kits may comprise one or more compositions which may be, but are not necessarily, complexed with a solid support or resin, such as antibodies; protein and peptide modifying reagents, such as proteases, kinases, or phosphatases; nucleic acids; compositions capable of covalently attaching themselves to proteins or peptides, such as fluorescent labels, radiolabels, and protecting groups; protein and peptide substrates or ligands; or any composition capable of being used for detecting or quantifying the amount of protein and peptide, nucleic acid, or other molecule(s) present in the sample. In a preferred embodiment, the additional reagent is an affinity chromatography resin. Such resins may include, but are not limited to, GST resins, nickel complex resins, resins with antibodies attached, ion-exchange resins, hydrophobic interaction resins, and the like. The additional reagents may be in the same container as the at least one pore containing matrix and cell lysing/disrupting/permeabilizing composition (FIG. 1), or in separate containers (FIGS. 3, 4 and 5). Such kits of the invention may also comprise collection tubes or receiver plates and protocols or instructions for carrying out the methods of the invention.

The invention also relates to an apparatus for use in extracting and isolating protein and peptide molecules comprising a container which comprises one or more compositions such as;

(a) at least one pore containing matrix, which retards the flow of high molecular weight molecules, structures and aggregates, but does not substantially retard the flow of soluble protein or peptide molecules in said container; and

(b) at least one composition selected from the group consisting of chromatographic resins that bind proteins or peptides, chromatographic resins that bind impurities, chromatographic resins having bound thereto protein modifying reagents, chromatographic resins having bound thereto enzymes, chromatographic resins having bound thereto nucleic acids, chromatographic resins having bound thereto an enzyme substrate, filters, and compositions capable of being used for detecting or quantifying the amount of protein or nucleic acid present in the sample.

In another preferred embodiment the invention relates to an apparatus for use in extracting and isolating protein and peptide molecules comprising a container which comprises one or more compositions such as;

(a) at least one pore containing matrix, which retards the flow of high molecular weight molecules, structures and aggregates, but does not substantially retard the flow of soluble protein or peptide molecules in said container; and

(b) at least one composition selected from the group consisting of antibodies which bind to the protein or peptides of the invention, substrates for said protein or peptides, ligands for said proteins or peptides, cofactors for said protein or peptides, nucleic acid molecules which bind to said proteins or peptides, inhibitors of said proteins or peptides, enzymes which modify said proteins or peptides, compositions which modify said proteins or peptides, compositions which bind said proteins or peptides, compositions which are bound by said proteins or peptides, and compositions capable of being used for detecting or quantifying the amount of protein or nucleic acid present in the sample.

Kits, compositions, apparatuses, and methods of the invention may also comprise any one, or combinations of, the components, compositions or apparatuses of the invention. More specifically, the kits of the invention may comprise one or more apparatuses of the invention, and one or more other composition described herein.

Other preferred embodiments of the present invention will be apparent to one of ordinary skill in light of what is known in the art, the following drawings and description of the invention, and the claims.

### BRIEF DESCRIPTION OF THE DRAWINGS/FIGURES

FIG. 1 is a diagram of one aspect of the invention, depicting a thin-walled tube (preferably a microfuge tube of any size) 1 containing a porous, matrix material in the form of a frit or plug or cartridge or swab tip 2 which divides the airspace within the tube into an upper sample application section 3 and a lower sample collection or sample elution section 4. According to one aspect of the invention, the matrix material 2 may comprise one or more cell lysing/disrupting/permeabilizing compounds or compositions. In another aspect, the matrix material may be in the form of beads or a gel or other semi-solid matrix in which case the matrix is preferably encased by, associated with, or supported by a solid support material 2a such as a frit or porous filter to maintain the upper sample-application section 3 and the lower sample collection section 4. Preferably, the matrix material (solid or semi-solid) is in the form of a cartridge or plug or swab tip which can be easily removed from the tube 1 to facilitate sample collection. In another aspect, one or more additional matrices or resins may be included in the upper sample application section 3 and/or in the sample-collection section 4, to further facilitate isolation or purification of the desired protein and peptide molecules. For example, well known protein and peptide binding matrices (such as ion-exchange resins, hydrophobic interaction resins, and affinity resins) may be included below a size separation matrix of the invention to further purify the desired protein and peptide molecules from undesired components including lipids, nucleic acids, lysis/disruption compositions used to lyse/disrupt the cellular source, solvents, detergents, etc. Alternatively, additional compositions which bind such undesired components but which do not substantially bind the desired protein and peptide molecules may be used. In another embodiment, combinations of such protein and peptide binding matrices and contaminant binding matrices may be used. The optional protein and peptide binding resin and/or contaminant binding resin 5 is shown. Such additional matrices may be in cartridge or plug or swab tip form. The optional protein and peptide binding resin or contaminant binding resin 5 may be encased by, associated with, or supported by a solid support material 5a such as a frit or porous filter. In another aspect, the sample-collection section 4 may contain an opening or access port (which may be closed if desired) to collect samples without the need to remove the matrix or matrices. In one example, where a size separation matrix and a protein or peptide binding matrix are provided, the desired protein and peptide molecules pass through the size separation matrix and bind to the binding matrix. Then suction can be applied to remove unwanted materials through the access port or opening within the sample-collection section 4. If desired, prior to the addition of the wash buffers, the size separation matrix may be removed from the tube 1. The desired isolated protein and peptide molecules may then be removed from the access port/opening when an elution buffer is applied. Alternatively, the removal of desired protein and peptide molecules is accomplished by removal of the matrix or matrices to access the sample-collection section 4.

FIG. 2 is a photograph of an ethidium bromide-stained 1% agarose gel, comparing Nsi I restriction endonuclease activity recovered by several cell extraction methods. Duplicate 1 μl aliquots of each sample were incubated with 0.6 μg lambda DNA. Lane 1, purified Nsi I control; lanes 2-3, sonicated sample; lanes 4-5, lysis matrix/filter matrix; lanes 6-7, Permneabilization Buffer only sample; lanes 8-9, lysis matrix/filter matrix without Permeabilization Buffer sample; lane 10, undigested lambda DNA control; and lane M, 1 Kb Plus DNA Ladder.

FIG. 3 is a diagram of one aspect of the invention, depicting a thin-walled tube or column (preferably microspin or spin cartridges of any size) 1 containing a lysis matrix/filter matrix 2 and a second tube or column containing an additional composition 5 for further purifying the smaller molecular weight protein and peptide molecules. Preferably, the additional composition 5 is a protein or peptide binding matrix or a contaminant binding matrix, or combinations thereof. The lysis matrix/filter matrix 2 and the additional composition 5 may be in close proximity and separated by a solid support material 2a such as a frit or porous filter; although, such matrices are preferably contained in separate tubes or columns 1. The tube or column 1 contains a sample application section 3 and an opening or access port 6 (which may be closed if desired) to collect the sample. An optional collection tube, well or container 7 is provided for collecting samples passing through the opening or access port 6. In a preferred aspect, the size separation matrix 2 comprises a cell lysis/disruption compound or composition. In the application of such preferred embodiment, a sample containing a cellular source of protein and/or peptide molecules are applied to the sample application section 3a preferably to the upper surface of the matrix 2. The cell lysis/disruption composition or compound causes release of the low and/or high molecular weight protein and peptide molecules which separate according to size in the size separation matrix 2, allowing protein and peptide molecules to pass through the matrix 2, while a substantial portion of the large molecular weight molecules and structures are retained in or on the matrix 2. Protein and peptide molecules passing through the size separation matrix 2 are channeled through the opening or access port, and into the sample application section 3b of a second tube or column containing a protein or peptide binding matrix 5. Eluted protein and peptide molecules then bind to the protein or peptide binding matrix 5. The size separation matrix 2 may optionally be removed from the column or tube 1 (before or after washing) to minimize large molecular weight molecules and structures from passing through the size separation matrix 2 during subsequent washing and elution. Washing buffers or solutions may then be applied to remove unwanted materials. An elution buffer or solution may then be applied to elute the desired protein and peptide molecules from the protein or peptide binding matrix and through the opening or access port 6. During washing, the collection tube 7 (containing the unwanted materials) can be replaced with a second or new collection tube 7 to collect the desired protein and peptide molecules upon elution.

FIG. 4 is a diagram of another aspect of the invention, depicting a thin-walled tube or column 1 containing a lysis matrix/filter matrix 2 on top of a solid support material 2a. The tube or column 1 contains a sample application section 3 and an opening or access port 6. A collection tube, well or container 7, containing a composition such as a protein binding matrix 5, is provided for collecting samples passing through the opening or access port 6. The composition 5 is supported by a solid support material 5a. This embodiment allows for the easy physical separation of the tube or column 1 containing the lysis matrix/filter matrix 2.

FIG. 5 is a diagram of another aspect of the invention, depicting a thin-walled tube or column 1 containing a lysis matrix/filter matrix 2 on top of a solid support material 2a. The tube or column 1 contains a sample application section 3 and an opening or access port 6. A collection tube, well or container 7, containing a composition such as a protein binding matrix in the form of beads 5, is provided for collecting samples passing through the opening or access port 6.

FIG. 6 is a photograph of an ethidium bromide stained 1% agarose gel comparing nucleic acid contamination in fractions recovered by several cell extraction methods. Duplicate 20 μl aliquots of each sample were analyzed by agarose gel electrophoresis. Lane 1, DNA extracted from cells using CloneChecker (Life Technologies, a division of Invitrogen Corp.); lanes 2-3, sonicated sample; lanes 4-5, lysis matrix/filter matrix; lanes 6-7, Permeabilization Buffer only sample; lanes 8-9, pore containing matrix without Permeabilization Buffer sample; lane 10, Permeabilization Buffer only control; and lane M, 1 Kb Plus DNA Ladder.

FIG. 7 is a scanned image of a stained SDS-PAGE gel comparing total protein recovery as well as protein recovery after secondary affinity tag purification of sonicated samples and samples isolated using the lysis matrix/filter matrix. Duplicate 15 μl aliquots of each sample were analyzed. Lane M, BenchMark Protein Ladder (Life Technologies, a division of Invitrogen Corp.); lanes 1-2, total protein from sonicated samples; lanes 3-4, total protein from the lysis matrix/filter matrix samples; lanes 5-6, sonicated samples after His-6 purification using Ni-NTA agarose beads (Qiagen); lanes 7-8, lysis matrix/filter matrix samples after secondary purification.

FIGS. 8A and 8B are scanned images of a stained SDS-PAGE gel comparing total protein recovery as well as protein recovery after secondary affinity tag purification of sonicated samples and samples isolated using the lysis matrix/filter matrix. Duplicate 15 μl aliquots of each sample were analyzed. FIG. 8A, samples isolated using sonication; Lane M, BenchMark Protein Ladder (Life Technologies, a division of Invitrogen Corp.); lanes 1-2, total protein; lanes 3-4, samples after secondary purification using GST purification (Amersham Biotech). FIG. 8B, samples isolated using the lysis matrix/filter matrix; Lane M, BenchMark Protein Ladder; lanes 1-2, total protein; lanes 3-4, samples after secondary purification.

FIG. 9 is a scanned image of a 1% TAE agarose gel. Lane M is a 1 kb Plus DNA ladder. Lambda DNA was restricted with NsiI, and the reaction products were run in lane 1 as a control. Lambda DNA was incubated with cellular extracts prepared by sonication (lane 2), and the methods of the invention (lanes 3 and 4), and the reaction products were run on the 1% TAE gel.

FIG. 10 is a scanned image of a 4-20% SDS page gel. The flow-through from the sample addition was run in lane “FLOW”, the eluted buffer from the column washing step was run in lane “WASH”, and lanes E1-E3 represent the eluates of three successive 100 μl elutions of the filter of the invention. 15 μl of each sample was added to each well of the gel.

FIG. 11 is a scanned image of a 4-20% SDS page gel. Lanes 1-3 show the purity and yield of a 20 kD insoluble protein isolated by the soluble method (lane 1), the insoluble method (lane 2), and the sonication/urea method (lane 3). Lanes 4-6 show the purity and yield of a 60 kD insoluble protein isolated by the soluble method (lane 4), the insoluble method (lane 5), and the sonication/urea method (lane 6). Lanes 7-9 show the purity and yield of a 120 kD insoluble protein isolated by the soluble method (lane 7), the insoluble method (lane 8), and the sonication/urea method (lane 9).

### DETAILED DESCRIPTION OF THE INVENTION

The present invention provides compositions, methods, and kits that may be used in extracting and isolating protein and peptide molecules from a protein and/or peptide containing cell. It will be readily appreciated by those skilled in the art that, in accordance with the present invention, any cell, tissues, organs, populations of cells, etc. can be used as a protein and peptide source.

In the description that follows, a number of terms used in the fields of molecular biology, biochemistry and protein chemistry are utilized extensively. In order to provide a clearer and consistent understanding of the specification and claims, including the scope to be given such terms, the following definitions are provided.

Pore. As used herein, the term pore refers to a single small space or opening in a matrix, which may be spherical, conical, elliptical, cylindrical or amorphous. In a preferred embodiment the pore is formed by the intersection of three or more fibers aligned or nearly aligned along the flow path. The average diameter of the pores of the matrix of the invention may range from about 0.1 to about 10,000 microns in diameter, about 0.1 to about 5,000 microns in diameter, about 0.1 to about 1,000 microns in diameter, about 1 to about 500 microns in diameter, about 10 to about 500 microns in diameter, or about 25 to about 400 microns in diameter.

High molecular weight molecule or structure. As used herein, the phrase is an arbitrary designation referring to any molecule or structure which is too large to freely pass through the pores of the selected matrix. It should be noted that the designation of a molecule or structure as “high molecular weight” can vary depending on the matrix selected. Examples of molecules and structures that would commonly be considered “high molecular weight” include, but are not limited to, chromosomal or genomic DNA, membrane fragments, liposomes, mitochondria, chloroplasts, ribosomes, or inclusion bodies (aggregates of molecules).

Host. Any prokaryotic or eukaryotic cell that produces the protein and/or peptide of interest. The terms “host” or “host cell” may be used interchangeably herein. For examples of such hosts, see Maniatis et al., “Molecular Cloning: A Laboratory Manual,” Cold Spring Harbor Laboratory, Cold Spring Harbor, N.Y. (1982). Preferred prokaryotic hosts include, but are not limited to, bacteria of the genus Escherichia (e.g., E. coli), Bacillus, Staphylococcus, Agrobacter (e.g., A. tumefaciens), Streptomyces, Pseudomonas, Salmonella, Serratia, Caryophanon, etc. The most preferred prokaryotic host is E. coli. Bacterial hosts of particular interest in the present invention include E. coli strains K12, DH10B, DH5α and HB101. Preferred eukaryotic hosts include, but are not limited to, fungi, fish cells, yeast cells, plant cells and animal cells. Particularly preferred animal cells are insect cells such as Drosophila cells, Spodoptera Sf9, Sf21 cells and Trichoplusa High-Five cells; nematode cells such as C. elegans cells; and mammalian cells such as COS cells, CHO cells, VERO cells, 293 cells, PERC6 cells, BHK cells and human cells. In accordance with the invention, a host or host cell may serve as the cellular source for the desired protein and/or peptide molecule to be isolated.

Native Conformation. As used herein, the term “native conformation” (as in native conformation and function) is defined as the tertiary or quaternary structure (or range of tertiary or quaternary structures) of the amino acid chain as it is known to exist in the biological host wherein the protein or peptide is naturally translated without intervention. It is generally assumed in the art, that a protein or peptide in its native conformation will also possess all native functions and activities. Perturbation of the native conformation often, but not necessarily, leads to perturbation of the native function or activity, such proteins and peptides could also be referred to as denatured proteins and peptides. The structure of proteins or peptides will be considered to be perturbed for the purposes of this application if their native structure cannot be regained without significant manipulation (e.g. remolding techniques). Proteins and peptides which substantially maintain their native conformations have substantially all of their native functions and activities.

Soluble protein. As used herein, the term “soluble protein” (as in small, soluble protein molecule) is defined as a protein molecule which, in its current conformation, is adequately surrounded by solvent molecules so as not to form large aggregates with other protein molecules in a non-specific manner (e.g. precipitation, floculation, etc). A contrasting term would be an insoluble protein to include transmembrane proteins, denatured proteins and proteins forming an inclusion body. Proteins or peptides which may be insoluble (form an inclusion body) in one solvent (e.g. an aqueous solvent), may be soluble in a different buffer system (e.g. 6M Urea).

Isolated. As used herein, the term “isolated” (as in “isolated protein molecule” or “isolated peptide molecule”) means that the isolated material, component, or composition has been at least partially purified away from other materials, contaminants, and the like which are not part of the material, component, or composition that has been isolated. For example, an “isolated protein molecule” is a protein molecule that has been treated in such a way as to remove at least some of the contaminants (e.g., membrane fragments or nucleic acids) with which it may be associated in the cell, tissue, organ or organism. As one of ordinary skill will appreciate, however, a solution comprising an isolated protein and/or peptide molecule may comprise one or more buffer salts, solvents, e.g., water, and/or other protein and peptide molecules, yet the desired protein and peptide molecules may still be considered an “isolated” protein and peptide molecules with respect to its starting materials.

Solubilization reagent, compound or composition. As used herein, solubilization reagent, compound or composition refers to a reagent, compound or composition that will effectively solubilize insoluble material (e.g. membrane fragments, inclusion bodies, etc). More specifically, the term refers to the ability to solubilize membrane fragments and/or inclusion bodies. Solubilize refers to the ability of a composition to disrupt aggregates, conglomerations or complexes of biological macromolecules (e.g. proteins), preferably by effectively surrounding the molecule with sufficient solvent molecules to prevent the molecule from forming aggregates with other protein molecules in a non-specific manner (e.g. precipitation, floculation, etc). Preferably, a solubilization composition, compound or reagent will solubilize at least about 25%, 50%, 75%, 80%, 85%, 90%, 95%, 97%, 99% or more of the total insoluble molecules of interest.

Cell lysing/disrupting/permeabilizing compound or composition. As used herein, “cell disrupting” or “cell lysing” refers to a composition or a component of a composition that effects lysis, rupture, or poration of the cells, tissues, or organisms used as the source of the protein and peptide molecules to be isolated, such that the soluble protein and peptide molecules (or portion thereof) that are contained in the cell, tissue, or organism source are released from the cell, tissue, or organism. According to the invention, the cells, tissues, or organisms need not be completely lysed/disrupted/permeabilized, and all of the protein and peptide molecules contained in the source cells, tissues or organisms need not be released therefrom. Preferably, a cell disrupting or cell lysis compound or composition will release at least 25%, 50%, 75%, 80%, 85%, 90%, 95%, 97%, 99%, or more of the total protein or peptide molecules of interest (soluble and insoluble) that are contained in the cell, tissue, or organism.

Other terms used in the fields of protein chemistry, biochemistry, recombinant DNA technology, molecular biology and cell biology as used herein will be generally understood by one of ordinary skill in the applicable arts.

### Sources of Proteins and Peptides

The methods, compositions and kits of the invention are suitable for isolation of protein and peptide molecules from any cellular source, including a variety of cells, tissues, organs or organisms, which may be natural or which may be obtained through any number of commercial sources (including American Type Culture Collection (ATCC), Rockville, Md.; Jackson Laboratories, Bar Harbor, Me.; Cell Systems, Inc., Kirkland, Wash.; Advanced Tissue Sciences, La Jolla, Calif.). Cells that may be used as cellular protein and peptide sources may be prokaryotic (bacterial, including members of the genera Escherichia particularly E. coli), Serratia, Salmonella, Staphylococcus, Streptococcus, Clostridium, Chlamydia, Neisseria, Treponema, Mycoplasma, Borrelia, Bordetella, Legionella, Pseudomonas, Mycobacterium, Helicobacter, Agrobacterium, Collectotrichum, Rhizobium, and Streptomyces) or eukaryotic (including fungi or yeasts, plants, protozoans and other parasites, and animals including humans and other mammals). Also suitable for use as sources of protein and peptide molecules are mammalian tissues or cells such as those derived from brain, kidney, liver, pancreas, blood, bone marrow, muscle, nervous, skin, genitourinary, circulatory, lymphoid, gastrointestinal and connective tissue sources (e.g. of endodermal or ectodermal origin), as well as those derived from a mammalian (including human) embryo or fetus. Appropriate sources of protein and peptide may also be any of the above cells harboring plasmids, phagemids, cosmids, viruses, phages, or other DNA molecules capable of expressing the desired proteins and peptides. These cells, tissues and organs may be normal, primary, transformed, or established cell lines, or they may be pathological such as those involved in infectious diseases (caused by bacteria, fungi or yeast, viruses (including AIDS) or parasites, in genetic or biochemical pathologies (e.g., cystic fibrosis, hemophilia, Alzheimer's disease, schizophrenia, muscular dystrophy or multiple sclerosis), or in cancers and cancerous processes. The methods, compositions and kits of the invention are well-suited for isolation of small soluble proteins and peptides, e.g. those of 1000 Kd or less, preferably, about 1-100 Kd, most preferably, about 1-50 Kd. One of ordinary skill in the art can choose a particular pore-containing matrix that will allow the isolation of proteins and peptides of any given molecular weight with no more than routine experimentation. The methods of the invention are particularly well suited for isolation of protein or peptide molecules expressed in a biological host, which form an inclusion body.

In a particularly preferred aspect, the methods of the invention are useful in the isolation of recombinant protein and peptide molecules expressed from DNA incorporated in a host capable of expressing said proteins and peptides. Particularly preferred protein and peptide molecules are part of a protein or peptide library. Such libraries include, but are not limited to populations of completely novel amino acid sequences encoded by random polynucleotide sequences, such as those which may be generated according to U.S. Pat. Nos. 5,763,192, 5,976,862, 5,824,514, 5,817,483, 5,814,476 and 5,830,721 or can be libraries or groups of randomly generated mutant proteins and peptides such as those of rho transcription termination protein generated by UV radiation (see Zweifka et. al., Biochemistry 32: 3564-70 (1993)). Other cells, tissues, viruses, organs and organisms that will be familiar to one of ordinary skill in the art may also be used as sources of protein and peptide molecules for the extraction and preparation of isolated protein and peptide molecules according to the present invention.

### Methods

In one aspect, the invention relates to methods for isolating protein and peptide molecules, particularly soluble protein and peptide molecules. Methods according to this aspect of the invention may comprise one or more procedures which result in the isolation of one or more protein and peptide molecules or populations of protein and peptide molecules (e.g., from a cDNA expression library) from the natural environment in which the protein and peptide molecules are found.

In one preferred such aspect, the methods of the invention may comprise:

(a) contacting one or more cellular sources of protein or peptide molecules, with at least one pore-containing matrix which substantially retards the flow of high molecular weight molecules, structures and aggregates, but does not substantially retard the flow of soluble protein and peptide molecules and causing the cellular source to release all or a portion of the desired soluble protein and peptide molecules; and

(b) separating or substantially separating the protein or peptide molecules from the high molecular weight molecules, structures and aggregates.

In another aspect of the invention, the invention relates to a method for obtaining one or more proteins and peptides comprising:

(a) contacting a cellular source of one or more proteins or peptides with at least one pore-containing matrix and causing the cellular source to release all or a portion of the one or more proteins or peptides; and

(b) separating or substantially separating the one or more desired proteins or peptides from undesired molecules obtained from said cellular source.

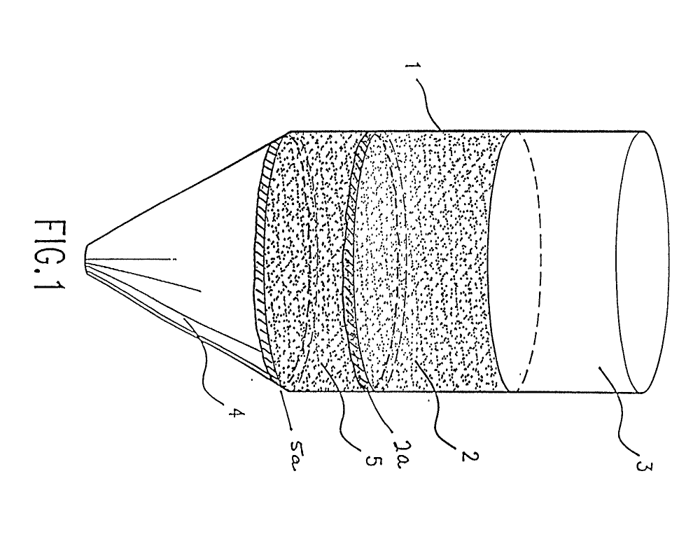

According to the invention, the matrix may be any porous matrix that substantially retards the flow (reversibly or irreversibly) of high molecular weight molecules, structures and aggregates but not substantially retard the flow of soluble protein and peptide molecules. Suitable materials for preparing the solid matrices used in this aspect of the invention include, but are not limited to, polyester, scintered polyethylene, nitrocellulose, polyolefin, cellulose acetate, nylon, cellulose, silica, and the like. This solid matrix may be provided in any convenient format for use in isolation of protein and peptide molecules, for example, as an insert (e.g., a frit or plug or swab or cartridge), as a membrane, as a filter, or as a densely packed porous matrix (e.g., beads or gels). In one aspect, for example, the matrix may be provided as a frit or cartridge or as a membrane suitable for insertion into a tube or column, providing a partitioning of upper and lower chambers of the tube or column by the matrix; such an aspect of the invention is diagramed in FIG. 1. The matrix may also be provided in other convenient forms, such as sheets, frits, plugs, cartridges or inserts suitable to fit multi-well plates typically used in filtration of multiple samples, including, for example, 6-well plates, 12-well plates, 24-well plates, 48-well plates, 96-well plates, 384-well plates, and the like, or suitable to fit into other plate sizes such as 35 mm plates, 60 mm plates, 100 mm plates, 150 mm plates, and the like. In a particularly preferred embodiment, the solid matrix is provided as a frit or insert or cartridge or swab suitable to fit into a microcentrifuge tube, microspin tube or spin cartridges. In one example, the frit/insert/cartridge/swab has a size of 8 mm diameter×1 cm length. Such tubes are available for example from NNI/Lida Manufacturing, Naperville, Ill.

The pores in the separation matrix are typically small enough to retard the flow of large molecules, structures and aggregates, but large enough to permit passage of soluble protein and peptide molecules, and may range from about 0.1 to about 10,000 microns in diameter, about 0.1 to about 5,000 microns in diameter, about 0.1 to about 1,000 microns in diameter, about 1 to about 500 microns in diameter, about 10 to about 500 microns in diameter, or preferably about 25 to about 400 microns in diameter. Larger or smaller pore sizes may also be used, provided the matrix is sufficiently dense so as to provide a “tortuous path” (as that phrase is commonly used by those of ordinary skill in the chromatography arts) preventing direct flow-through of the large molecular weight molecules and structures, but still permitting flow-through of the soluble protein and peptide molecules.

In preferred use, the cellular source is applied onto the matrix, preferably in an aqueous solution, and then is introduced into or on the matrix either by unit gravity incubation or preferably by centrifugation or vacuum. The cellular source will optionally be trapped within or on the matrix in preparation for release of the protein and peptide molecules. Lysis/disruption/permeabilization compositions, physical forces and/or mechanical forces (or combinations thereof) may be used for disrupting the integrity of the cell membrane/cell wall of the cellular source of the protein and peptide molecules. In accordance with the invention, any physical or mechanical forces (freezing, heating, freeze-thawing, pressure, sonication etc.) may be used separately or in combination with the lysis/disrupting/permeabilizing compounds or compositions to release the desired protein and peptide molecules from the cellular source. Preferably, the matrix comprises such lysis/disruption compounds or compositions. According to the invention, the lysis/disruption composition or compound may be either applied to the matrix containing the cellular source or preferably may be adsorbed, complexed or associated with (e.g., by ionic, hydrophobic, covalent or non-covalent binding) the matrix prior to applying the cellular source to the matrix, for example by soaking or saturating the matrix in the lysing/disrupting/permeabilizing composition and then, optionally, allowing the matrix to dry under air, vacuum and/or heat; alternatively, the composition may be applied to the matrix material just prior to its use or prior to the preparation of the matrix plug, frit, insert, membrane, etc. from the matrix material. Any method of pre-treating the pore-containing matrix results in the formation of a matrix that has been impregnated with a lysing/disrupting/permeabilizing composition. Thus, in a preferred aspect, the matrix comprises the lysis/disruption/permeabilization compositions or compounds. In this preferred aspect of the invention, contacting of the cellular source and the lysis/disrupting/permeabilizing of the present invention are thus accomplished concurrently or nearly concurrently, thereby reducing the amount of time and manipulation required for the extraction of the protein and peptide molecules.

In one preferred embodiment, an effective amount of the composition that disrupts the cellular membrane/cell wall integrity that is applied to the matrix, or that is pre-adsorbed onto the matrix, may comprise one or more detergents, which may be a non-ionic detergent, including, but not limited to, N-octyl-β-D-glucopyranside, N-octyl-β-D-maltoside, ZWITTERGENT 3.14, deoxycholate; n-Dodecanoylsucrose; n-Dodecyl-β-D-glucopyranoside; n-Dodecyl-β-D-maltoside; n-Octyl-β-D-glucopyranoside; n-Octyl-β-D-maltopyranoside; n-Octyl-β-D-thioglucopyranoside; n-Decanoylsucrose; n-Decyl-β-D-maltopyranoside; n-Decyl-β-D-thiomaltoside; n-Heptyl-β-D-glucopyranoside; n-Heptyl-β-D-thioglucopyranoside; n-Hexyl-β-D-glucopyranoside; n-Nonyl-β-D-glucopyranoside; n-Octanoylsucrose; n-Octyl-β-D-glucopyranoside; n-Undecyl-β-D-maltoside; APO-10; APO-12; Big CHAP; Big CHAP, Deoxy; BRIJ® 35; C₁₂E₅; C₁₂E₆; C₁₂E₈; C₁₂E₉; Cyclohexyl-n-ethyl-β-D-maltoside; Cyclohexyl-n-hexyl-β-D-maltoside; Cyclohexyl-n-methyl-β-D-maltoside;Digitonin; ELUGENT™; GENAPOL® C-100; GENAPOL® X-080; GENAPOL® X-100; HECAMEG; MEGA-10; MEGA-8; MEGA-9; NOGA; NP-40; PLURONIC® F-127; TRITON® X-100; TRITON® X-114; TWEEN® 20; or TWEEN® 80. Additionally, the detergent may be an ionic detergent, including, but not limited to, BATC, Cetyltrimethylammonium Bromide, Chenodeoxycholic Acid, Cholic Acid, Deoxycholic Acid, Glycocholic Acid, Glycodeoxycholic Acid, Glycolithocholic Acid, Lauroylsarcosine, Taurochenodeoxycholic Acid, Taurocholic Acid, Taurodehydrocholic Acid, Taurolithocholic Acid, Tauroursodeoxycholic Acid, and TOPPA. Zwitterionic detergents can also be used with the compositions and methods of the invention, including, but not limited to, amidosulfobetaines, CHAPS, CHAPSO, carboxybetaines, and methylbetaines.

The concentration of the detergent may be from about 0.01 to 10% by weight, 0.01 to 5% by weight, 0.01 to 4% by weight, 0.01 to 3% by weight, 0.01 to 2.5% by weight, 0.1 to 10% by weight, 0.1 to 5% by weight, 0.1 to 4% by weight, 0.1 to 3% by weight, 0.1 to 2.5% by weight, 0.5 to 10% by weight, 0.5 to 5% by weight, 0.5 to 4% by weight, 0.5 to 3% by weight, 0.5 to 2.5% by weight, 1.0 to 10% by weight, 1.0 to 5% by weight, 1.0 to 4% by weight, 1.0 to 3% by weight or 1.0 to 2.5% by weight. Most preferably the detergent concentration is 2.5%. In addition, one or more enzymes such as lysozyme, lyticase, zymolyase, neuraminidase, streptolysin, cellulysin, mutanolysin, chitinase, glucalase or lysostaphin may be used, at a concentration of about 0.1 to 5 mg/ml; one or more inorganic salts such as sodium chloride, potassium chloride, magnesium chloride, calcium chloride, lithium chloride, or praseodymium chloride at a concentration of about 1 mM to 5M; or any other compound which disrupts the integrity of (i.e., lyses or causes the formation of pores in) the membrane and/or cell wall of the cellular source of protein and peptide molecules (e.g., polymixin B), or combinations of the foregoing may be used. The compositions may also comprise other components, such as protease inhibitors (e.g., phenylmethylsulfonyl fluoride, trypsin inhibitor, aprotinin, pepstatin A), reducing reagents (e.g., 2-mercaptoethanol and dithiothreitil) at concentrations of 0.1 to 10 mM, chelating agents (e.g., disodium ethylenediaminetetraacetic acid (Na₂EDTA), EGTA, CDTA, most preferably at a concentration of about 1 mM or less) and/or one or more ribonucleases (RNase A, T1, T2, and the like) at concentrations ranging from 1 to 400 μg/ml, or any combination of the foregoing. DNase I concentrations may range from 1 to 100 units (10,000 units/mg). In one preferred embodiment, the composition provides for the disruption of the cell membrane or cell wall integrity without substantially perturbing the native conformation or function of the desired proteins and peptides, so that a protein or peptide having the native conformation, or substantially the native conformation may be collected. However, if the native structure of the protein or peptide is not required, then no limitation on the lysis/disruption reagent is required. The lysis/disruption compositions preferably comprises less than 10% cell lysis/disruption/permeabilization composition, more preferably, less than 5% cell lysis/disruption/permeabilization composition and most preferably, less than 3% cell lysis/disruption/permeabilization composition. A most preferred composition comprises 2.5% ELUGENT™, Calbiochem Corporation (San Diego, Calif.). In other embodiments of the invention, the ELUGENT™ concentration may range from about 0.01 to 10% by weight, 0.01 to 5% by weight, 0.01 to 4% by weight, 0.01 to 3% by weight, 0.01 to 2.5% by weight, 0.1 to 10% by weight, 0.1 to 5% by weight, 0.1 to 4% by weight, 0.1 to 3% by weight, 0.1 to 2.5% by weight, 0.5 to 10% by weight, 0.5 to 5% by weight, 0.5 to 4% by weight, 0.5 to 3% by weight, 0.5 to 2.5% by weight, 1.0 to 10% by weight, 1.0 to 5% by weight, 1.0 to 4% by weight, 1.0 to 3% by weight or 1.0 to 2.5% by weight. Desired concentrations and combinations of the active ingredients of the lysis/disruption compositions may be readily determined by those skilled in the art. In another aspect of the invention, cell lysis/disruption and disruption/solubilization of insoluble material can be accomplished with one composition or reagent that serves both functions.

Once the cellular source of protein and peptide molecules has been contacted with the matrix and the cells lysed/disrupted/permeabilized, the protein and peptide molecules contained within the cellular source are released from the cell and the high molecular weight molecules, structures and aggregates are bound to or trapped within or on the matrix material, while the soluble protein and peptide molecules substantially pass through the matrix material without being substantially bound thereby or trapped therein. These soluble protein and peptide molecules may be collected with the flow-through, for example by washing the matrix with an aqueous solution sufficient to wash or elute the soluble protein and peptide molecules through the matrix, but insufficient to remove the large molecules and structures from the matrix to which they are bound or in which they are trapped. In another aspect of the invention, the cells or cellular source can be lysed before or after being contacted with the lysis matrix/filter matrix of the invention.

In another preferred embodiment, after cell lysis or disruption, insoluble material (e.g., membrane fragments and/or inclusion bodies) may be trapped in the matrix of the invention. Such insoluble material may be associated with the matrix after the soluble protein has been eluted from the matrix. The matrix may then be contacted with a second elution reagent which is capable of disrupting the membrane fragments or inclusion bodies, and solubilizing the proteins contained therein. These protein and peptide molecules can then be collected with the flow-through, for example by washing the matrix with an amount of solution sufficient to wash or elute the soluble protein and peptide molecules through the matrix.

In accordance with the invention, the desired protein and peptide molecules obtained may be further purified by well known protein and peptide purification or chromatography techniques. In a preferred embodiment, such further purification procedures may involve affinity chromatography (e.g., nickel or GST resins), ion-exchange chromatography, hydrophobic interaction chromatography, precipitation (e.g., with PEI, PEG or ammonium sulfate) and the like. Thus, the invention further comprises purifying the desired protein and peptide molecules by any known techniques available in the art. In a particularly preferred embodiment of the invention, the compositions used in the further purification procedures (e.g. resins, antibodies, etc) are present in the collection container of the invention, such that after the proteins or peptides isolated by the methods of the invention are eluted from the matrix they will pass into, or be added to, the collection container which contains these compositions for further purification. Such additional purification may facilitate removal of unwanted contaminants such as nucleic acids, other proteins and peptides, lipids, nucleotides, oligonucleotides, or compounds or compositions which may inhibit the activity of or further manipulation of the protein and peptide molecule (e.g., labeling, cleaving via proteolysis, detection and quantitation of enzyme activity, etc). In any event, such further purification need not take place and thus the protein and peptide molecules obtained by the method of the invention may be manipulated directly by standard biochemistry and protein chemistry techniques. In a preferred aspect of the invention, one or more additional purification compositions (e.g., ion exchange resins, affinity resins, magnetic beads, antibodies, nickel resins, GST resins, etc) are utilized in combination with the separation matrix in accordance with the invention. Such additional purification may be accomplished in separate procedures, although in a preferred aspect, the additional purification is accomplished simultaneously or in conjunction with the separation method of the invention. In one aspect, the one or more separation matrices and the one or more protein and peptide purification compositions are associated in series, in a fluid channel, such that a sample containing the desired protein and peptide molecules may pass from one matrix to another. In this aspect, the separation matrix and purification composition combination may be provided in any format to provide a fluid channel to associate the various matrices in fluid connection such as a column format, a tube format, a well format, a multi-well plate format, etc. In this embodiment, the desired protein and peptide molecules passing through the separation matrix would subsequently contact the protein or peptide purification composition. In one embodiment of the invention, removal of unwanted materials (such as lipids, nucleic acids, lysis/disruption compositions, and components which may inhibit further manipulation or analysis of protein and peptide molecules) are removed with a wash buffer or solution which allows the desired protein and peptide molecules to be retained on the immobilized purification composition. An elution buffer or solution for removing the desired protein and peptide molecules from the immobilized purification composition may then be used to isolate the purified protein and peptide molecules.

In a highly preferred embodiment the invention can be used for screening libraries of protein and peptide molecules in a high throughput format. For example, a library of random or mutated polynucleotide sequences, such as those generated in U.S. Pat. No. 5,763,192, may be screened for enzymatic activity or binding properties in a 96 well plate, using the described invention. Colonies of bacteria, each containing a plasmid encoding one member of the library, may be applied to the matrix after induction of protein or peptide synthesis. The cells containing the protein or peptide are then lysed/disrupted/permeabilized. Protein and peptide molecules are then eluted from the matrix using a buffered aqueous solution and/or centrifugation and collected in the wells of a 96 well plate. Reagents containing desired ligands or substrates may also be present in the 96 well plate, and presence of activity or binding may then be measured by any methods deemed appropriate for the activity or binding properties desired.

In another preferred embodiment the invention can be used for screening libraries of randomly or systematically generated mutants of a particular protein or peptide of interest. Preliminary evidence demonstrated a library of mutants of reverse transcriptase could be screened efficiently for relative enzymatic activity using the 96-well lysis matrix/filter matrix plate. Additionally, screening can be accomplished by immobilizing the proteins or peptides of the invention onto a substrate, such as a multi-well plate, chip, slide, wafer, filter, sheet, tube, and the like. These substrates, containing the immobilized protein or peptides of the invention, can be contacted with a composition that either binds to protein or peptide molecules (e.g. antibodies), is bound by the protein or peptide molecules (e.g., ligands) or causes a change in a measurable parameter (e.g. luminescence, color change, fluorescence, chemiluminescence, etc.).

### Compositions

In a related aspect, the invention relates to compositions for use in isolating protein and/or peptide molecules. Compositions according to this aspect of the invention may comprise one or more components or portions, such as:

(a) one or more cellular sources of the desired protein or peptide molecules;

(b) at least one pore-containing matrix which substantially retards the flow of high molecular weight molecules, structures and aggregates, but does not substantially retard the flow of soluble protein or peptide molecules; and optionally

(c) at least one compound or composition that disrupts or lysis one or more cells of the cellular source.

Preferred such cellular sources, matrices, and compounds and compositions for use in the compositions of the invention include those described and used in the methods of the present invention. In a preferred composition of the invention, the matrix comprises the compound that disrupts the integrity of the cellular membrane or cell wall. An effective amount of such compound is preferably adsorbed onto or complexed with or associated with the matrix, for example by ionic, hydrophobic, non-covalent or covalent attachment of the lysis/disrupting compound or composition to the matrix material. The compositions of the invention are useful in isolating a variety of protein and peptide molecules, particularly those described herein and most particularly recombinant, proteins and peptides from bacterial cells, expressed either as soluble proteins or in an inclusion body.

The invention also relates to an apparatus for use in extracting and isolating protein and peptide molecules comprising a housing which comprises one or more compositions such as;

(a) at least one pore containing matrix, which substantially retards the flow of high molecular weight molecules, structures and aggregates, but does not substantially retard the flow of soluble protein or peptide molecules in said container; and

(b) at least one composition selected from the group consisting of chromatographic resins that bind proteins or peptides, chromatographic resins that bind impurities, chromatographic resins having bound thereto protein modifying reagents, chromatographic resins having bound thereto enzymes, chromatographic resins having bound thereto nucleic acids, chromatographic resins having bound thereto an enzyme substrate, filters, and compositions capable of being used for detecting or quantifying the amount of protein or nucleic acid present in the sample.

Examples of chromatographic resins that bind proteins or peptides include resins having bound thereto antibodies, protein ligands, compositions capable of covalently attaching themselves to the protein or peptides, and the like.

In another preferred embodiment the invention relates to an apparatus for use in extracting and isolating protein and peptide molecules comprising a housing which comprises one or more compositions such as;

(a) at least one pore containing matrix, which substantially retards the flow of high molecular weight molecules, structures and aggregates, but does not substantially retard the flow of soluble protein or peptide molecules in said container; and

(b) at least one composition selected from the group consisting of antibodies which bind to the protein or peptides of the invention, substrates for said protein or peptides, ligands for said proteins or peptides, cofactors for said protein or peptides, nucleic acid molecules which bind to said proteins or peptides, inhibitors of said proteins or peptides, enzymes which modify said proteins or peptides, compositions which modify said proteins or peptides, compositions which bind said proteins or peptides, compositions which are bound by said proteins or peptides, and compositions capable of being used for detecting or quantifying the amount of protein or nucleic acid present in the sample.

The apparatus of the invention may fisher comprise:

(c) a porous solid support disposed between the at least one pore containing matrix and any additional compositions; and/or

(d) a sample application section and a sample collection section, separated by the pore containing matrix.

### Kits

In another embodiment, the invention relates to kits for use in isolating protein and peptide molecules. Such kits of the invention may comprise one or more components, which may be contained in or include one or more containers such as boxes, cartons, tubes, microspin tubes, microfuge tubes, spin cartridges, multi-well plates, vials, ampules, bags, and the like. In one such aspect, the kits of the invention may comprise one or more of the compositions of the invention described in detail herein. In another aspect, the kits of the invention may comprise:

(a) at least one matrix which (which is preferably contained in a tube, column, cartridge, well etc.) substantially retards the flow of high molecular weight molecules, structures and aggregates, but does not substantially retard the flow of soluble protein and/or peptide molecules; and

(b) a cell lysing/disrupting/permeabilizing composition or compound.

In one such kit, the matrix comprises an effective amount of the cell lysing/disrupting/-permeabilizing composition or compound which may be adsorbed onto or complexed with or associate with the matrix, for example by ionic, hydrophobic, non-covalent or covalent attachment of the composition or compound to the matrix material. In another aspect, the kits comprise additional protein and/or peptide purification compositions, wash buffers, elution buffers etc. Preferred matrix materials, cell lysis/disrupting/permeabilizing compositions and compounds, and elution and wash compositions for use in the kits of the invention include those described herein for use in the methods and compositions of the present invention.

The kits of the invention may further comprise one or more additional components or reagents that may be useful in further processing, analysis, or use of the protein and peptide molecules isolated or purified according to the invention, for example components or reagents useful in protein and peptide purification, labeling, or detection. Such reagents or components may, for example, include one or more resins which bind amino acid sequences to aid in purification (e.g., nickel resins, and GST binding resins), or other reagents that will be familiar to one of ordinary skill in the art.

### Isolated Protein and Peptide Molecules

The invention also relates to isolated protein and peptide molecules that are prepared according to the methods of the invention. In one preferred embodiment, the isolated protein and peptide molecules of the invention are recombinant, proteins and peptides, particularly those expressed in and isolated from bacterial cells.

In a related aspect, the invention provides the ability to quickly screen and evaluate recombinant proteins and peptides prepared by recombinant technologies (e.g., by cloning and expression). The invention thus may be used to quickly isolate such recombinant proteins and peptides, providing a ready source of the recombinant proteins and peptides for such evaluation or screening (e.g., by analysis of enzyme activity, analysis of binding properties, ability to be bound by a specific antibody, etc.). The invention further relates to immobilizing the protein or peptide molecules of the invention on a solid substrate for the purpose of high throughput screen. Examples of such solid substrates include, but are not limited to, multi-well plates, chips, slides, wafers, filters, sheets, tubes, and the like. Proteins or peptides immobilized on appropriate substrates can then be screened by any method known in the art, including but not limited to, hybridization with an antibody, contacting with a substrate, contacting with a ligand, contacting with a biological macromolecule (e.g. DNA, RNA, protein, peptide, carbohydrate, lipid, amino acid, nucleotide, nucleoside, etc.) and the like. The proteins or peptides immobilized on the substrate can be analyzed for the presence of an appropriate signal, which may include, but is not limited to, fluorescence, chemiluminescence, bioluminescence, absorption of a particular wavelength of light, binding of a particular substrate, changes in color, or any other method deemed appropriate to gain the information desired.

The invention also relates to the use of recombinant host cells comprising the isolated protein and peptide molecules of interest, the use of such cells to isolate such proteins and peptides produced according to the invention, and recombinant protein and peptide molecules of the invention. Representative host cells (prokaryotic or eukaryotic) that may be used according to the invention include, but are not limited to, bacterial cells, yeast cells, plant cells and animal cells. Such suitable host cells are available commercially, for example from Life Technologies, a division of Invitrogen Corp. (Rockville, Md.), ATCC (Manassas, Va.), and other commercial sources that will be familiar to one of ordinary skill in the art. Host cells comprising the proteins and peptides, recombinant proteins and peptides or isolated protein and peptide molecules of the invention may be prepared by inserting DNA molecules or vectors containing genes encoding a protein or peptide of interest into the host cells, using well-known transformation, electroporation, infection or transfection techniques that will be familiar to one of ordinary skill in the art. According to this aspect of the invention, introduction of the DNA molecules into a host cell capable of producing the desired protein or peptide from the inserted DNA, can be accomplished by any known method of introducing nucleic acid molecules into host cells, including but not limited to calcium phosphate transfection, DEAE-dextran mediated transfection, cationic lipid-mediated transfection, electroporation, transduction, transformation (e.g., of competent cells particularly E. coli cells), infection or other methods. Such methods are described in many standard laboratory manuals, such as Davis et al., “Basic Methods In Molecular Biology” (1986) and Maniatis et al., “Molecular Cloning: A Laboratory Manual”, Cold Spring Harbor Laboratory, Cold Spring Harbor, N.Y. (1982). Appropriate culture media and cultivation conditions for the transformed or transfected host cells are known in the art. Methods for expressing proteins and peptides from recombinant DNA molecules introduced into appropriate hosts are well known to one of ordinary skill in the art.

### Uses of Isolated Protein and Peptide Molecules

The protein and peptide molecules isolated by the compositions, methods and kits of the present invention may be further characterized or manipulated, for example by labeling, protease digestion, analysis of enzymatic or binding activity and the like.

Alternatively, protein and peptide molecules isolated according to the present invention may be used for the manufacture of various materials in industrial processes by methods that are well-known in the art. Such materials include, but are not limited to, pharmaceuticals (enzymatic catalysis of pharmaceutical precursors); protein and peptide molecular weight standards; modification of proteins and peptides, DNA, lipids or carbohydrates by enzymatic catalysis and the like. Additionally libraries of expressed protein and peptide molecules may be screened in a high throughput format using a multi-well plate (e.g. 96 well, 384 well, etc) for the presence of a desired characteristic or activity.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Isolation of Protein and Peptide From Bacterial Cells

The aim of this project was to improve the process of extracting protein from bacterial cells. Specifically, the objectives were first, to develop a more rapid lysis procedure where there are fewer manipulations and the manipulations are more forgiving, and second, to eliminate a separate centrifugation or filtration procedure for the removal of membrane fragments and cell debris. According to the present invention, these objectives are accomplished by integration of the lysis and filtration processes into a single operation. The output from this operation is soluble protein ready for further purification, if necessary, by matrix chromatography. By the present invention, it is further possible to combine the matrix chromatography procedure with the lysis and precipitate removal procedure, to make a single unit operation of the entire protein preparation method.

### Materials and Methods

Unless otherwise noted, all procedures were performed at room temperature, and all reagents are from Life Technologies, a division of Invitrogen Corp., Rockville, Md.

96-Well lysis matrix/filter matrix. One 19.2 mm circumference×10.0 mm long filter plug of bonded polyester fiber was placed into a single well of a 96-well filter plate containing a glass fiber membrane (GF/F) (Cat. No. 7700-2810, Polyfiltronics/Whatman, Rockland, Mass.). The plug filter was force-fit into the well, so it rested very near the bottom of the well, just above (0-1 mm) the GF/F membrane. Snugness of fit within the well was important so that all liquid was forced through the plug filter, and not between the well side and the plug filter side. In a like manner, the remaining wells of the 96-well filter plate were installed with plug filters.

Cell Growth. E. coli DH5α mcr rec⁺ harboring plasmids ptrcNsiI 215 and pSURpslNsiI 191 (Fermentation Seed #657, Life Technologies, a division of Invitrogen Corp., Rockville, Md.) was grown for 16 hrs at 37° C. at 250 rpm in Circle Grow medium (Cat. No.3000-122, Bio101, Inc., Vista, Calif.) supplemented with 100 μg/ml ampicillin and 50 μg/ml kanamycin. One milliliter of the overnight culture was used to inoculate a fresh 30-ml aliquot of the same medium with antibiotics. Growth of the diluted culture continued at 37° C. with shaking at 250 rpm. Cell growth was monitored by turbidity at 600 nm. Once the culture reading of O.D.₆₀₀ was 0.704, protein over-expression was induced by addition of 300 μl of 100 mM IPTG to the medium. Growth continued at 37° C. for another 2 hrs at 250 rpm. Final cell density was O.D.₆₀₀=1.3.

Protein Extraction by 96-Well lysis matrix/lysis matrix. A 200-μl aliquot of the induced culture was applied directly to the plug filter surface of the 96-Well lysis matrix/filter matrix. A duplicate sample was applied to a second filter. After 10 min, 100 μl of Permeabilization Buffer (0.3 M Bis-Tris, pH 7.0, 30 mM EDTA, 15% (v/v) Triton X-100, 6% (w/v) deoxycholic acid) were added to both filters. Incubation continued for 10 min, then the 96-Well lysis matrix/filter matrix plate was aligned on top of a 96-well, 800-μl receiver plate (Cat. No. 7701-1800, Polyfiltronics/Whatman, Rockland, Md.) and centrifuged 5 min at 3000×g in a swinging bucket rotor. Collected volumes were transferred to individual 1.5-ml microcentrifuge tubes and placed at +4° C.

As a control for the efficacy of the Permeabilization Buffer, duplicate 200-μl aliquots of the induced culture were applied to separate plug filters of the 96-Well lysis matrix/filter matrix as before. However, in this case, no Permeabilization Buffer was added. After 20 min, the 96-Well lysis matrix/filter matrix plate was centrifuged as before. Recovered volumes were transferred to individual 1.5-ml microcentrifuge tubes and placed at +4° C.

Protein Extraction with Buffer Only. Duplicate 200-μl aliquots of the induced culture were placed in 1.5-ml microcentrifuge tubes. To each sample was added 100 μl of Permeabilization Buffer, vortexed for 2 s, then allowed to stand 10 min. The tubes were centrifuged at 12,000×g for 10 min. Supernatants were transferred to individual 1.5-ml microcentrifuge tubes and placed at +4° C.

Protein Extraction by Sonication. Duplicate 200-μl aliquots of the induced culture were placed in separate 1.5-ml microcentrifuge tubes. Both tubes were centrifuged for 10 min at 12,000×g to collect the cells. After removing the supernatants, the cell pellets were each suspended in 300 μl of 50 mM Bis-Tris, pH 7.0, 10 mM EDTA. The cell suspensions were each subjected to three 10-s pulses from a Sonic Dismembrator (Model 550, Fisher Scientific), using a microtip submerged in the liquid at a setting of “3”. Both tubes were centrifuged for 10 min at 12,000×g to collect the debris. Supernatants were transferred to individual 1.5-ml microcentrifuge tubes and placed at +4° C.

Enzyme Activity Assay. Nsi I restriction endonuclease activity was detected by a standard assay with lambda DNA. An aliquot (1 μl) of each sample to be tested was added to 0.6 μg lambda DNA (Life Technologies, a division of Invitrogen Corp.) and 1× React 3 Buffer in a total volume of 20 μl. As a positive enzyme control, 1 μl (10 Units) of purified Nsi I (Life Technologies, a division of Invitrogen Corp.) was used in place of the sample. As a negative enzyme control, 1 μl of water was substituted for the sample addition in the reaction. All reaction mixtures were mixed briefly, then incubated at 37° C. for 1 hr. Reactions were terminated by addition of one-tenth volume of Endo R Stop Solution (0.1 M EDTA (pH 8.0), 0.1% (w/v) bromphenol blue, 1% SDS, and 50% (v/v) glycerol).

Agarose Gel Electrophoresis. Aliquots were subjected to electrophoresis through a 1% w/v) agarose gel in TAE buffer at 100 VDC. The 1 Kb Plus DNA Ladder (Life Technologies, a division of Invitrogen Corp.) was run in parallel as a molecular size standard. DNA was detected by ethidium bromide staining, followed by photography under UV transillumination.

### Results and Discussion

Several procedures were performed to determine whether lysis matrix/filter matrix could form the basis of a simplified process to extract proteins from bacterial cells. The procedure could be most useful if recovered proteins maintained their native conformation. An indication of the gentleness of the technique could be made by measurement of activity of model enzymes. A further advantage would be gained if extracting the sample using lysis matrix/filter matrix simultaneously provided a simple purification procedure.

In earlier work with Lysis matrix for isolation of plasmid DNA (see U.S. application No. 09/478,456), the buffer used in cell lysis contained strong protein denaturants. Such an extraction buffer would not be appropriate here, especially when native, active protein is sought; thus, milder buffer conditions should be used. A buffer containing a non-denaturing detergent was used for the methods described herein to permit immediate assay of the recovered enzyme without further processing.

Bacterial cells harboring plasmids encoding Nsi I restriction endonuclease were cultured in liquid medium under conditions that induced over-production of the protein. Several samples of the culture were subjected in parallel to different protein extraction methods for comparison. As a first method, cells were harvested from a sample of the culture and suspended in buffer. The suspension was sonicated in order to disrupt physically the cell membranes, causing release of the cells' contents, including the protein of interest. In a second method, similar to the procedure cited for MMLV-RT, a culture sample was mixed with Permeabilization Buffer, incubated, and then centrifuged to remove most insoluble debris. In a third method, samples from the cell culture were applied directly to the surface of a plug filter in the 96-Well Lysis matrix/filter matrix plate. Once the cells entered the plug filter, a one-half volume of Permeabilization Buffer was added to the surface of the plug filter. Protein extraction occurred in the interior of the plug filter. Centrifugation of the Lysis matrix/filter matrix plate passed soluble material through the depth of the plug filter and the small pore (ave. 0.7 μ) glass fiber membrane and into the well of the receiver plate.

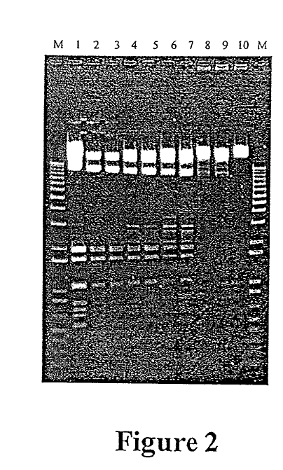

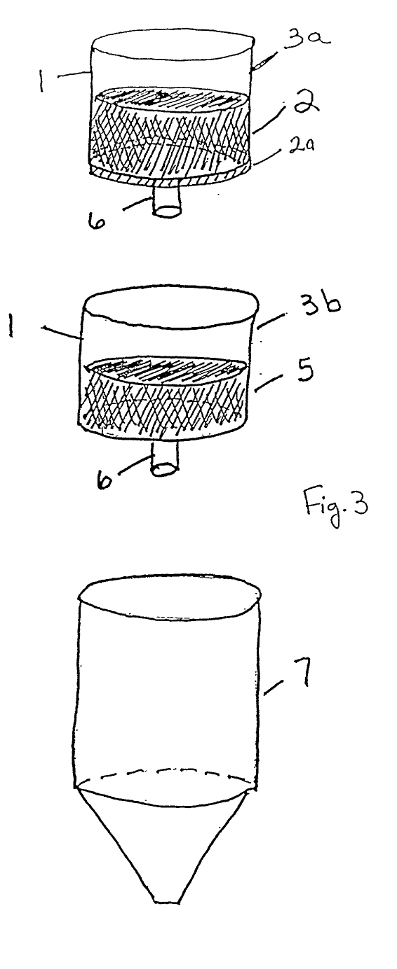

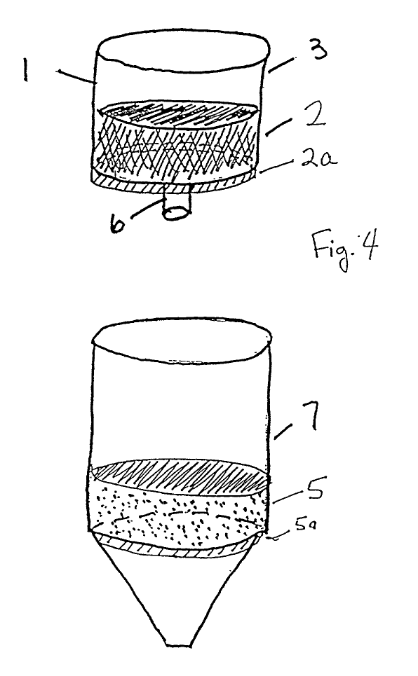

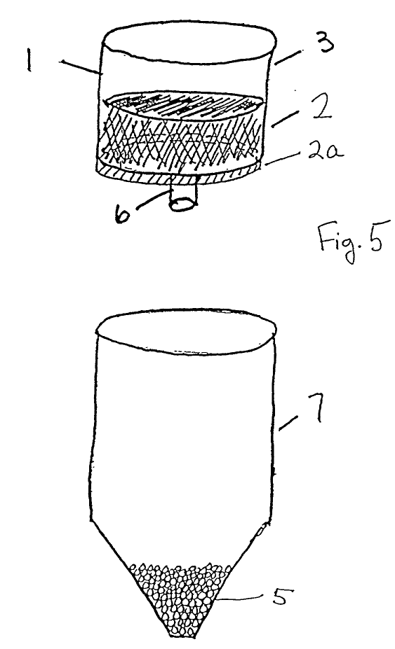

An assay designed to measure specifically Nsi I activity is used first to establish whether active protein is extracted. Restriction endonuclease activity degrades lambda DNA into a number of discretely sized fragments, causing a unique pattern or fingerprint. In FIG. 2 is shown an agarose gel of the restriction endonuclease assay performed on samples extracted by several methods. An authentic fragmentation pattern is seen in lane 1 for reference. Lanes 6 and 7 is seen significant Nsi I activity, demonstrating that Permeabilization Buffer did extract active protein. Using Lysis matrix/filter matrix with Permeabilization Buffer (lanes 4 and 5) also extracted active enzyme. On the other hand, samples processed through the Lysis matrix/filter matrix without Permeabilization Buffer (lane 8 and 9) showed very little Nsi I endonuclease. The small amount of activity observed is probably from some cells that lysed/disrupted/permeabilized during handling. Lanes 2 and 3 confirm significant active Nsi I endonuclease is obtained when the traditional method of sonication is used.

Careful observation of the fragmentation patterns in FIG. 2 reveal a qualitative measure of the amounts of enzyme extracted by the several methods. When little enzyme was recovered, as in lanes 8 and 9, most of the lambda DNA remained intact, appearing as a prominent band similar in mobility to that of the no enzyme control (lane 10). Full activity is seen in the sonicated sample, where no intact lambda DNA or partially digested fragments, best observed in the 2 to 10 Kb size range, are present. Significant, but less than full activity, is seen in the samples extracted with Permeabilization Buffer.

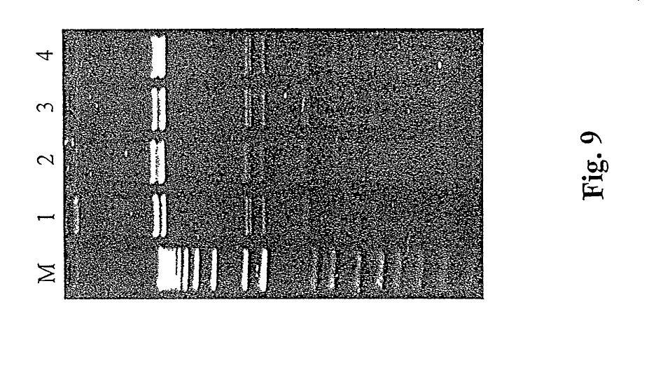

A similar procedure was performed using the 96 well embodiment of the invention. Cells expressing NsiI were cultured to an OD₆₀₀ of 2.0. 200 μl of the culture was added to the matrix, followed by 100 μl of lysis buffer. A second aliquot of the cells was subjected to sonication. Aliquots of the sonicated extract and the extract lysed by the methods of the invention, were incubated with lamda DNA and the samples were then run on a 1% agarose gel. As seen in FIG. 9, both the sonicated sample (lane 2) and the samples prepared by the methods of the invention (lanes 3 and 4) showed evidence of significant NsiI activity; however, the sonicated sample (lane 2) suggests a large amount of nucleic acid contamination in the preparation, probably due to genomic DNA shearing during sonication.

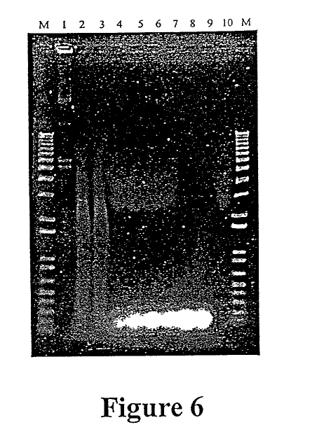

Lysis matrix/filter matrix is shown in FIGS. 2 and 9 to extract protein and maintain enzyme activity. From direct observation of the samples processed, no debris pellet was obtained when samples were processed by the Lysis matrix/filter matrix, whereas a significant pellet was recovered from the sonication method. Use of Permeabilization Buffer without Lysis matrix/filter matrix also showed a substantial pellet. To examine the purification procedure further, samples from each extraction method were electrophoresed directly on an agarose gel shown in FIG. 6. The ethidium bromide staining of the gel will assay for nucleic acid contamination in the sample. A heavy smear of fluorescence is seen in the sonicated samples (lanes 2 and 3), indicating random shearing of genomic DNA and rRNA, and a failure to separate nucleic acids from extracted proteins. Only a bright band of low molecular weight is seen in lanes 4-9, indicative of tRNA and small RNA fragments. The fuzzy light area about midpoint in lanes 4-7 and 10 is due to a component in Permeabilization Buffer, and of no consequence. Thus, protein extraction with Lysis matrix/filter matrix provides an added benefit of a significant purification, removing debris and most of the nucleic acids, while reducing the number of processing procedures.

Lysis matrix/filter matrix is a simpler and more powerful protein extraction procedure than commercially available products, such as BugBuster™ and B-PER. Since genomic DNA does not appear in samples from Lysis matrix/filter matrix, there are no sample viscosity problems to overcome with separate digestion with Benzonase® Nuclease as is the case with BugBuster™. In addition, maintaining most of the nucleic acids within the cell when Permeabilization Buffer is used, provides a lower background for enzymes used in molecular biological procedures. Furthermore, using Lysis matrix/filter matrix retains cell membranes, separating them and many biomolecules away from the soluble extracted proteins.

### Example 2

### Isolation and Subsequent Affinity Tag Purification of Protein From Bacterial Cells

With the method for isolation of proteins using only a single step for lysis and filtration established, this method was developed further to incorporate subsequent purification (as with an affinity tag). In this example, the loading of the purified protein onto the affinity matrix was done as a secondary process but could be carried out in a single procedure along with lysis and filtration. Additional modifications were made to the method and buffer systems to maximize protein yield and streamline the processing steps.

### Materials and Methods

Unless otherwise noted, all procedures were performed at room temperature, and all reagents are from Life Technologies, a division of Invitrogen Corp., Rockville, Md.

Cell Growth. E. coli BL21-SI harboring plasmid pEXP15-GUS (Gateway clone 6His-Gus) for Ni-NTA methods or pEnterGUS (Gateway clone GST-GUS) for GST methods, Life Technologies, a division of Invitrogen Corp., Rockville, Md. was grown for 16 hrs at 30° C. at 250 rpm in LBON medium supplemented with 100 μg/ml ampicillin. Three milliliter of the overnight culture was used to inoculate a fresh 30-ml aliquot of the same medium with antibiotics. Growth of the diluted culture continued at 30° C. with shaking at 250 rpm. Cell growth was monitored by turbidity at 600 nm. Once the culture reading of O.D.₆₀₀ was 0.600-0.800, protein over-expression was induced by addition of 1.8 milliliters of 5M NaCl to the medium. Growth continued at 30° C. for another 3 hrs at 250 rpm. Final cell density was O.D.₆₀₀=1.3-2.1.

Protein Extraction by 96-Well Lysis matrix/filter matrix. Duplicate 1.3 ml aliquots of the induced culture were placed in separate 1.5-ml microcentrifuge tubes. Both tubes were centrifuged for 10 min at 12,000×g to collect the cells. After removing the supernatants, the cell pellets were resuspended in 200 μl Resuspension Buffer (50 mM phosphate, pH 8.0, 30 mM KCl, 0.15% (v/v) Triton X-100) and incubated on ice for 10 min (this incubation step gives the highest yield but is not absolutely necessary). After the 10 min incubation on ice, 200 μl of resuspension was applied directly to the plug filter surface of the 96-Well Lysis matrix/filter matrix. A duplicate sample was applied to a second filter. 100 μl of Lysis Buffer (150 mM phosphate pH 8.0, 300 mM KCl, 1.5% (v/v) Triton X-100, 1.5 mg/ml lysozyme) were added to both filters. Incubation continued for 10 min, then the 96-Well Lysis matrix/filter matrix plate was aligned on top of a 96-well, 650-μl receiver plate (Cat. No. p9605, Labnet International) and centrifuged 5 min at 700-1000×g in a swinging bucket rotor. Collected volumes were transferred to individual 1.5-ml microcentrifuge tubes and placed at +4° C.

Protein Extraction by Sonication. Duplicate 1.3 ml aliquots of the induced culture were placed in separate 1.5-ml microcentrifuge tubes. Both tubes were centrifuged for 10 min at 12,000×g to collect the cells. After removing the supernatants, the cell pellets were each suspended in 300 μl of 50 mM phosphate pH 8.0, 300 mM NaCl. The cell suspensions were each subjected to three 10-s pulses from a Sonic Dismembrator (Model 550, Fisher Scientific), using a microtip submerged in the liquid at a setting of “3”. Both tubes were centrifuged for 10 min at 12,000×g to collect the debris. Supernatants were transferred to individual 1.5-ml microcentrifuge tubes and placed at +4° C.

Affinity Purification by Ni-NTA Agarose Beads (Qiagen Catalog number 31314) The NTA-Ni agarose beads were equilibrated with of 50 mM phosphate pH 8.0, 100 mM KCp, 0.15% Triton X-100 as a 50% slurry. Duplicates of 250 μl of total protein extracted by filterplate method and sonication method were incubated with 100 μl of 50% slurry Ni-NTA agarose beads in a 1.5 ml microcentrifuge tube. The samples were incubated with the agarose beads for 10 min and then centrifuged for 2 min at 700×g. The beads were washed twice with 1 ml of 50 mM phosphate pH 8.0, 300 mM NaCl, 25 mM imidizol, 0.5% glycerol centrifuged for 2 min at 700×g. The Poly-His tagged protein was eluted from the beads by incubating for 10 min with 200 μl of 50 mM phosphate pH 8.0, 300 mM NaCl, 500 mM imidizol, 10% glycerol, centrifuged for 2 min at 700×g and the eluate was collected in a 1.5-ml microcentrifuge tube and placed at +4° C.

Affinity Purification with MicroSpin GST Purification Module Affinity (Pharmacia Biotech, Inc. catalog number 27-4570-03). Duplicates of 250 μl of total protein extracted by filterplate method and sonication method were loaded onto the Glutathione SEPHAROSE 4B MicroSpin Column, gently mixed, and incubated for 10 min. The column was centrifuged for 1 min at 700×g, and the flow through was discarded. The column was washed twice with 1×PBS (Life Technologies, A division of Invitrogen Corp.) and centrifuged for 1 min at 700×g. The GST tagged protein was eluted from the column by incubating for 10 min with 200 μl of 10 mM glutathione, 50 mM Tris-HCl pH 8.0. The eluate was collected in a 1.5-ml microcentrifuge tube by centrifugation for 2 min. at 700×g.

SDS-PAGE Analysis Fifteen microliter aliquots of total protein and eluate of each sample were subjected to electophoresis through a 4-20% Tris-Glycine Gel (Novex) in 1×TGS Buffer (Life Technologies, a division of Invitrogen Corp. catalog number 15556-020) The BenchMark protein marker (Life Technologies, a division of Invitrogen Corp. catalog number 10747-012) was run in parallel as a molecular size standard. The proteins were detected by staining with Gel CodeBlue Stain Reagent (Pierce catalog number 24592).

### Results and Discussion

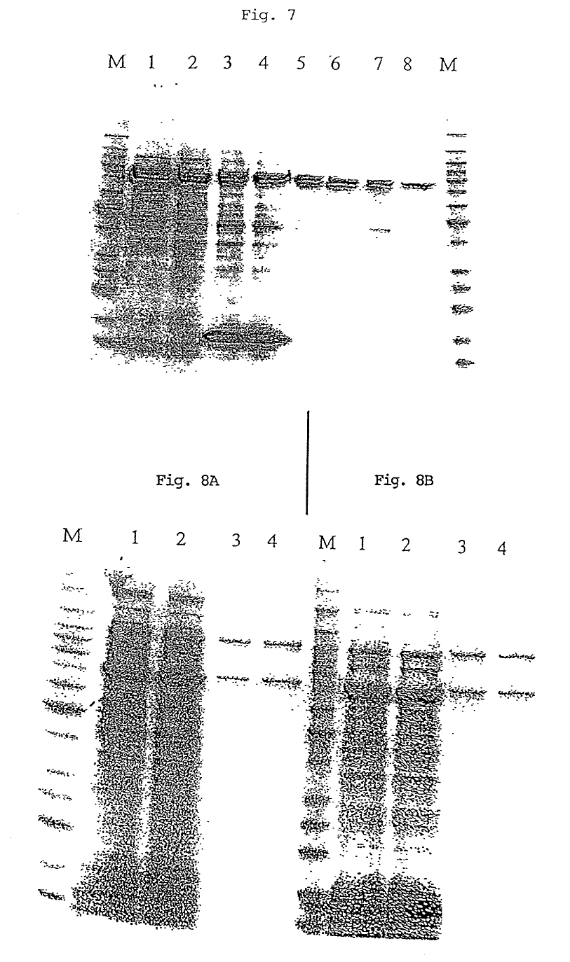

For adequate protein yield to allow quantitative recovery from secondary purification steps such as affinity resin based capture the use of lysozyme or other cell disruption methods was found to be useful. These types of cell disruption methods are also useful when using cells with tough membranes. The buffer system was also modified to allow compatibility with direct loading onto secondary purification schemes such as Ni-NTA or GST matricies. FIG. 7 shows that purification of total protein from plasmid pEZ15974 using the method of the invention (lanes 3 and 4) is at least equal to the total protein obtained by sonication (lanes 1 and 2). The additional band near the bottom of the gel in lanes 3 and 4 is contributed by the lysozyme protein. When identical total protein samples were further purified via the His 6 affinity tag using a commercially available system again, results for the samples purified by the inventions (lanes 7 and 8) were approximately equal in yield to those purified from sonicated samples (lanes 5 and 6).

FIGS. 8A and 8B show similar results from protein purified from plasmid pEnterGUS, which contains a GST fusion. FIG. 8A shows proteins obtained by sonication as the primary method of purification, lanes 1 and 2 contain total protein and lanes 3 and 4 are the same samples post GST purification. Similar results are seen in FIG. 8B, where the total protein purified using the method of the invention is shown in lanes 1 and 2 while lanes 3 and 4 show the samples after additional GST purification.

### Example 3

### Tandem Lysis-Capture and Affinity Tag Purification of Protein From Bacterial Cells

As described above, subsequent purification step can be performed either separately or in tandem with the lysis-capture procedure. In this procedure a hexahistidine tagged protein was purified in a tandem lysis-capture/affinity tag purification procedure to demonstrate the feasability of this approach.

### Materials and Methods

Unless otherwise noted, all procedures were performed at room temperature, and all reagents are from Life Technologies, a division of Invitrogen Corp., Rockville, Md.

Cell Growth. Cell culture conditions were identical to those in Example 2.

Protein Extraction by 96-Well Lysis matrix/filter matrix. One milliliter of the induced culture was placed in separate 1.5 ml microcentrifuge tubes. The tube was centrifuged for 10 min at 12,000×g to collect cells. After removing the supernatant, the cell pellet was resuspened in 200 μl of Resuspension Buffer (50 mM sodium phosphate pH 8.0, 100 mM KCl, 0.5%(v/v) Triton X-100) and incubated on ice for 10 min(this step is not necessary). After 10 min incubation on ice, 200 μl of resuspension was applied directly to the filterplate surface of the 96-well Lysis matrix/filter matrix. 100 μl of Lysis Buffer (150 mM sodium phosphate, 300 mM KCL, 1.5% (v/v) triton X-100, 1.5 mg/ml lysozyme) was added to the filter. Incubation continued for 10 min, then the 96-Well Lysis matrix/filter matrix was aligned on top of a SwellGel™Nickel Chelating Disc, 96-Well Filter Plate (Pierce Cat. No. 75824). The stack was placed on top of a 96 well, 650 μl receiver plate (Cat. No. p9605, Labnet International) and centrifuged 10 min at 500×g in a swinging bucket rotor. The follow-through was collected and transferred to 1.5 ml microcentrifuge tube and placed at +4° C. After centrifugation, the beads were washed once with 250 μl of 50 mM sodium phosphate pH 8.0, 300 mM NaCl, and 40 mM imidizol and centrifuged 10 min at 500×g in a swinging bucket rotor. The poly-his tagged fusion protein was eluted from the beads by incubating for 5 min with 100 μl of 50 mM sodium phosphate pH 8.0, 300 mM NaCl, and 250 mM imidizol and centrifuged 10 min at 500×g in a swinging bucket rotor. The elution step was repeated twice.

Protein Extraction by Sonication. One milliliter of the induced culture was placed in separate 1.5 ml microcentrifuge tubes. The tube was centrifuged for 10 min at 12,000×g to collect cells. After removing the supernatant, the cell pellet was resuspened in 300 μl 50 mM sodium phosphate pH 8.0, 100 mM KCl. The cell suspension was subjected to three 10-s pulses from a Sonic Dismembrator (Model 550, Fisher Scientific), using a microtip submerged in the liquid at a setting of “3”. The tube was centrifuged for 10 min at 12,000×g to collect the debris.

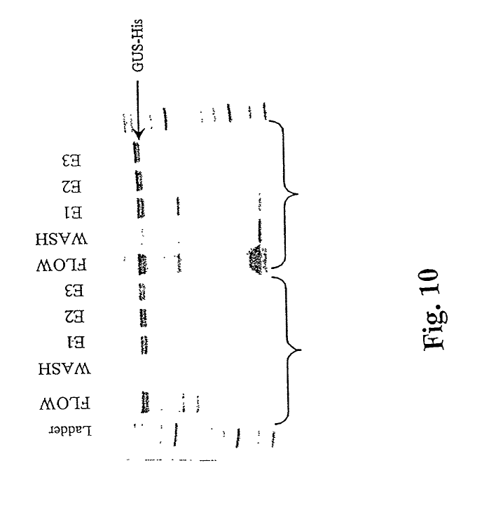

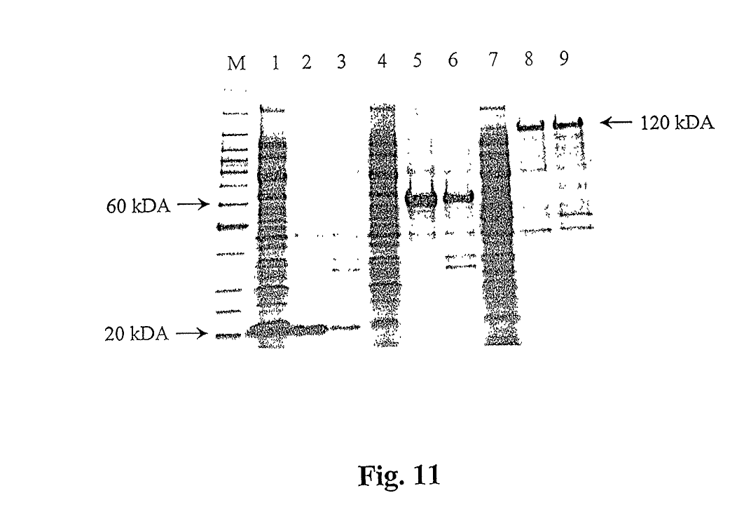

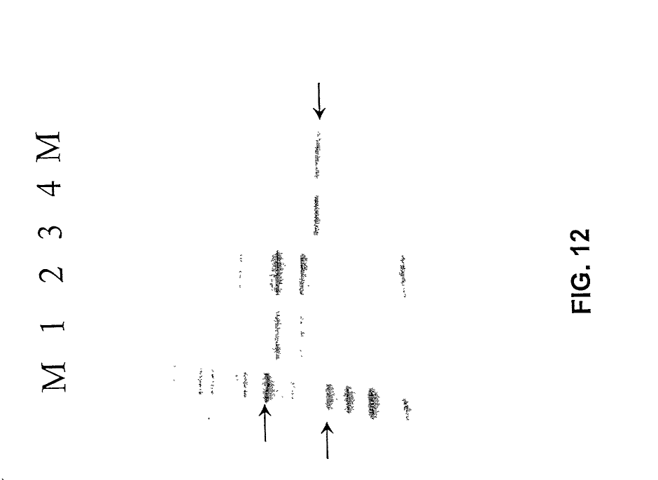

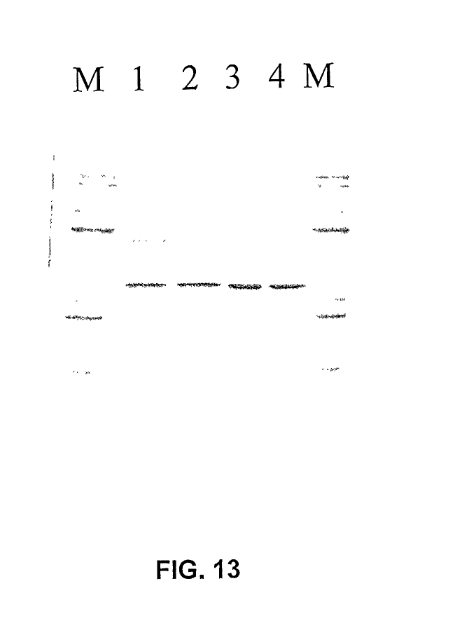

Affinity Purification by Ni-NTA Agarose Beads. The supernatant was transferred to 96 well SwellGel™Nickel Chelating Disc, 96-Well Filter Plate (Pierce Cat. No. 75824) and then placed on top of a 96 well, 650 μl receiver plate (Cat. No. p9605, Labnet International) and centrifuged 10 min at 500×g in a swinging bucket rotor. The follow-through was collected and transferred to 1.5 ml microcentrifuge tube and placed at +4° C. After centrifugation, the beads were washed once with 250 μl of 50 mM sodium phosphate pH 8.0, 300 mM NaCl, and 40 mM imidizol and centrifuged 10 min at 500×g in a swinging bucket rotor. The poly-his tagged fusion protein was eluted from the beads by incubating for 5 min with 100 μl of 50 mM sodium phosphate pH 8.0, 300 mM NaCl, and 250 mM imidizol and centrifuged 10 min at 500×g in a swinging bucket rotor. The elution step was repeated twice and 15 μl aliquots of each elution were loaded on a 4-20% SDS gele for PAGE analysis (FIG. 10).

SDS-PAGE Analysis. Fifteen microliters of the follow-through, the wash and the eluate of each sample were subjected to electrophoresis through a 4-20% Tris-Glycine Gel (Invitrogen Corporation, Cat. No. EC60252) in 1×TGS Buffer (Life Technologies, a division of Invitrogen Corporation, Cat No. 15556-020). The Benchmark protein marker (Life Technologies, a division of Invitrogen Corp. Cat No. 10747-012) was run in parallel as a molecular size standard. The proteins were detected by staining with Gel CodeBlue Stain Reagent (Pierce Cat No. 24592).

### Results and Discussion

As FIG. 10 shows, the tandem lysis-capture and affinity-tag purification resulted in a highly purified preparation of the fusion protein. With each successive elution the purity of the fusion protein increased (lanes E1-E3).

### Example 4

### Purification of Insoluble Proteins

The compositions and methods of the invention are compatible with the purification of both soluble and insoluble protein. The following procedures were developed to demonstrate the utility of the invention in isolating insoluble proteins, for example proteins which, when expressed, form an inclusion body.

### Materials and Methods

Unless otherwise noted, all procedures were performed at room temperature, and all reagents are from Life Technologies, a division of Invitrogen Corp., Rockville, Md.

Cell Growth. E.coli DH10B harboring plasmid pTRXFUSPRL20B (Benchmark protein clone 20 kDa) and E. coli stain STBL-2 harboring plasmids pTRXFUSPRL60B and pTRXFUSPRL120B (Benchmark protein clones 60 kDa and 120 kDa) was grown 16 hours at 30° C. at 250 rpm in Circle Grow medium supplemented with 100 μg/ml ampicillin. One half milliliter of overnight culture was used to inoculate 30-ml of Circle Grow medium supplemented with 100 μg/ml ampicillin. Growth of the diluted mixture continued at 30° C. with shaking at 250 rpm. Cell growth was monitored by turbidity at 600 nm. Once the culture reading of O.D.₆₀₀ was 1.0-1.2, raising the incubation temperature to 42° C. induced protein overexpression. Growth continued at 42° C. for 30 min and then at 37° C. for 1.5 hours. The final cell density was O.D.₆₀₀ was 2.0.

Protein Extraction by 96-Well Lysis matrix/filter matrix. One milliliter of the induced culture was placed in separate 1.5 ml microcentrifuge tubes. The tube was centrifuged for 10 min at 12,000×g to collect cells. After removing the supernatant, the cell pellet was resuspened in 200 μl of Resuspension Buffer (50 mM sodium phosphate pH 8.0, 100 mM NaCl, 0.5%(v/v) Triton X-100, 1.5% (v/v) NOG). The 200 μl of resuspension was applied directly to the filterplate surface of the 96-well Lysis matrix/filter matrix and incubated for 10 min at room temperature. 100 μl of Lysis Buffer (150 mM sodium phosphate, 300 mM NaCl, 1.5% (v/v) triton X-100, 1.5 mg/ml lysozyme) was added to the filterplate. Incubation continued for 10 min, then the 96-Well Lysis matrix/filter matrix was aligned on top of a 96 well, 650 μl receiver plate (Cat. No. p9605, Labnet International) and centrifuged 10 min at 1000×g in a swinging bucket rotor. Soluble protein was collected in the receiver plate and the inclusion bodies were trapped in the matrix. The matrix was then washed with 500 μl of ddH₂O and centrifuged for 5 min. at 1000×g. The wash was discarded. The 96 Well Lysis matrix/filter matrix plate was aligned on top of another 96-well, 650 μl receiver plate (Cat. No. p9605, Labnet International).

Protein Extraction by Sonication. One milliliter of the induced culture was placed in separate 1.5 ml microcentrifuge tubes. The tube was centrifuged for 10 min at 12,000×g to collect cells. After removing the supernatant, the cell pellet was resuspened in 300 μl of 50 mM sodium phosphate pH 8.0, 100 mM KCl. The cell suspension was subjected to three 10-s pulses from a Sonic Dismembrator (Model 550, Fisher Scientific), using a microtip submerged in the liquid at a setting of “3”. The tube was centrifuged for 10 min at 12,000×g to collect the debris. The cell pellet was washed 3 times with one milliliter of ddH₂O. The inclusion body pellet was then solubilized with 300 μl of 50 mM sodium phosphate pH 8.0, 8 M urea, 100 mM NaCl.

Addition of Second Elution/Disruption Reagent. 300 μl of Insoluble Buffer (150 mM sodium phosphate pH 8.0, 8M urea, 300 mM NaCl) was added to the filter and incubated for 10 min at room temperature. The plate was then centrifuged at 1000×g for 5 min in a swinging bucket rotor, and the solubilized protein was collected in the collection plate.

### Results

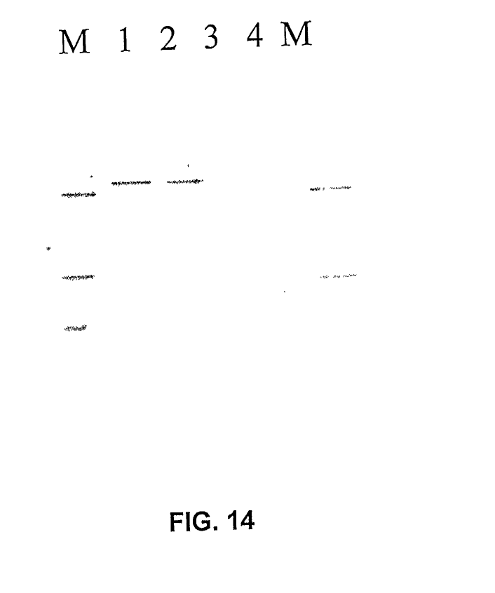

FIG. 11 shows the isolation of insoluble proteins of three sizes, 20 kD, 60 kD and 120 kD. Lanes 1, 4 and 7 show the soluble fractions which were eluted from the filter of the invention, for the 20, 60 and 120 kD proteins respectively. From these lanes it is clear that there is very little protein present in the soluble fraction. The amount of protein in the soluble fraction of the 20 kD protein is higher due to the partial solubility of this protein. Lanes 2, 5 and 8 show the eluate of the 20, 60 and 120 kD proteins respectively, after the addition of the second elution/disruption reagent. These lanes show a marked increase in the protein yield over similar procedures using sonication (lanes 3, 6 and 9). As such, the methods and compositions of the invention are very useful in isolating proteins which, when expressed, form an inclusion body. The methods appear to generate higher yields than similar methods using sonication.

### Example 5

### Purification of Insoluble Proteins by Direct Load Method

### Methods

Cell Growth. Cell culture conditions were identical to Example 2.

Protein Extraction by 96 well Lysis matrix/filter matrix. Duplicate 200 μl aliquots of culture were applied directly to the filter surface of the 96-Well Lysis matrix/filter matrix. 100 ul of Lysis Buffer (150 mM sodium phosphate pH 8.0, 300 mM NaCl, 2%(v/v) ELUGENT™, 1.5%(v/v) Triton X-100, 0.025 mg/ml lysozyme) were added to both filters. Incubation continued for 10 min at room temperature, then the 96 Well Lysis matrix/filter matrix plate was aligned on top of a 96-well, 650 ul receiver plate (Cat. No. p9605, Labnet International) and centrifuged 5 min at 1000×g in swing bucket rotor. Soluble protein was collected in the receiver plate and the inclusion bodies were trapped in the matrix. The matrix was then washed with 500 μl of ddH₂O and centrifuged for 5 min. at 1000×g. The wash was discarded. The 96 Well Lysis matrix/filter matrix plate was aligned on top of another 96-well, 650 ul receiver plate (Cat. No. p9605, Labnet International). 300 ul of Insoluble Buffer (150 mM sodium phosphate pH 8.0, 8M urea, 300 mM NaCl) was added to the filter and incubated for 10 min at room temperature. The plate was then centrifuged at 1000×g for 5 min in a swinging bucket rotor, and the solubilized protein was collected in the collection plate.

SDS PAGE Analyis: Fifteen microliters of the soluble fraction and 15 μl of the insoluble faction were subjected to electrophoresis through a 4-20% Tris-Glycine Gel (Invitrogen Corp. Cat No. EC60252) in 1×TGS Buffer (Life Technologies, a division of Invitrogen Corporation, Cat No. 15556-020). The Benchmark protein marker (Life Technologies, a division of Invitrogen Corporation, Cat No.10747-012) was run in parallel as a molecular size standard. The proteins were detected by staining with Gel CodeBlue Stain Reagent (Pierce Cat No. 24592).

### Results

FIG. 12 shows the isolation of insoluble protein of 35 kDa. Lanes 2 and 3 show the soluble fractions and Lanes 4 and 5 show the insoluble fractions. From these lanes it is clear that there is very little of the 35 kDa protein present in the soluble fraction. Lane 1 is Benchmark Protein ladder.

### Example 6

### Isolation and Subsequent Affinity Tag Purification of Protein From Bacterial Cells by Direct Load Method

### Methods

Cell Growth. Cell culture conditions were identical to Example 2

Protein Extraction by 96 well Lysis matrix/filter matrix. Duplicate 200 μl aliquots of induced culture were applied directly to the filter surface of the 96-Well Lysis matrix/filter matrix. 100 μl of Lysis Buffer (150 mM sodium phosphate pH 8.0, 300 mM NaCl, 2%(v/v) ELUGENT™, 1.5%(v/v) Triton X-100, 0.025 mg/ml lysozyme) were added to both filters. Incubation continued for 10 min at room temperature, then the 96 Well Lysis matrix/filter matrix plate was aligned on top of a 96-well, 650 μl-receiver plate (Cat. No. p9605, Labnet International) and centrifuged 5 min at 1000×g in swing bucket rotor. Soluble protein was collected in the receiver plate.

Affinity purification by Ni-NTA Agarose Beads. (Qiagen catalog number 31314). The Ni-NTA agarose beads were equilibrated with 50 mM sodium phosphate pH8.0, 100 mM NaCl, 0.15% triton X-100 as a 50% slurry. Duplicated of 250 μl of total protein extracted by the filterplate method were incubated with 50 μl of 50% slurry Ni-NTA agarose beads in a 1.5 ml microcentrifuge tube. The samples were incubated for 10 min and then centrifuges for 2 min at 700×g. The beads were washed 3 times with 1 ml 50 mM sodium phosphate pH 8.0, 300 mM NaCl, 20 mM imidizol centrifuged at 700×g. The poly-his tagged protein was eluted from the beads by incubating for 10 min with 50 μl of 50 mM sodium phosphate pH 8.0, 300 mM NaCl, 500 mM imidizol, centrifuged for 2 min at 700×g and the eluate was collected in a 1.5 ml microcentrifuge tube and placed at +4° C.

Affinity Purification with MicroSpin GST purification Module. (Pharmacia Biotech, Inc. Cat No. 27-45670-03). Duplicates of 250 μl of total protein extracted by the filterplate method were loaded onto the Glutathione SEPHAROSE 4B Microspin column, gently mixed, and incubated for 10 min. The column was centrifuged for 1 min at 700×g, and the follow-through discarded. The column was washed 3 times with 1×PBS (Life Technologies, a division of Invitrogen Corporation) and centrifuged for 1 min. at 700×g. The GST tagged protein was eluted from the column by incubating for 10 min with 50 μl of 10 mM glutathione, 50 mM Tris-HCl pH 8.0. The eluate was collected in a 1.5 ml microcentrifuge tube by centrifugation for 2 min at 700×g and place at +4° C.

SDS PAGE Analysis. Fifteen microliters of total protein and eluate of each sample were subjected to electrophoresis through a 4-20% Tris-Glycine Gel (Invitrogen Corporation, Cat No. EC60252) in 1×TGS Buffer (Life Technologies, a division of Invitrogen Corporation, Cat No. 15556-020). The Benchmark protein marker (Life Technologies, a division of Invitrogen Corporation, Cat No. 10747-012) was run in parallel as a molecular size standard. The proteins were detected by staining with Gel CodeBlue Stain Reagent (Pierce Cat No. 24592).

### Results

In FIG. 13, lane 1 shows Benchmark Protein Ladder. Lane 2 and 3 is total protein of 30 kDa poly his tagged fusion protein. Lane 4 and 5 is 30 kDa poly-his tagged fusion protein purified by Ni-NTA agarose beads.

In FIG. 14, lane 1 shows Benchmark Protein Ladder. Lane 2 and 3 is total protein of 58 kDa GST tagged fusion protein. Lane 4 and 5 is 58 kDa GST tagged fusion protein purified by MicroSpin GST purification.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

What is claimed is:

1. A method for isolating a protein molecule or population of protein or peptide molecules, comprising:
(a) contacting one or more cellular sources of protein or peptide molecules with at least one pore-containing matrix which substantially retards the flow of high molecular weight molecules, structures, and aggregates but does not substantially retard the flow of soluble protein and peptide molecules;
(b) separating or substantially separating said molecules from said high molecular weight molecules and structures.

2. The method of claim 1, further comprising causing the cellular source to release all or a portion of the said protein or peptide molecules.

3. The method of claim 1, wherein said matrix is selected from the group consisting of a polyester matrix, a polyolefin matrix, a sintered polyethylene matrix, a nitrocellulose matrix, a cellulose acetate matrix, a nylon matrix, a cellulose matrix and a silica matrix.

4. The method of claim 1, wherein the average size of said pores in said matrix range from about 1,000 microns to about 0.1 microns in diameter.

5. The method of claim 4, wherein said pores are from about 500 to about 1 microns in diameter.

6. The method of claim 5, wherein said pores are from about 400 to about 25 microns in diameter.

7. The method of claim 1, wherein said release of the said protein or peptide molecules are accomplished by a lysis/disruption/permeabilization composition or compound.

8. The method of claim 7, wherein said lysis/disruption/permeabilization composition comprises one or more detergents.

9. The method of claim 7, wherein said lysis/disruption/permeabilization composition comprises one or more enzymes.

10. The method of claim 9, wherein said enzyme is lysozyme, lysostaphin or zymolyase.

11. The method of claim 1, wherein said matrix comprises one or more lysis/disruption/permeabilization compositions or compounds.

12. The method of claim 1, further comprising
(a) contacting said filter with a composition that disrupts and/or solubilizes protein aggregates and/or membrane fragments;
(b) collecting the solubilized or disrupted protein or peptide molecules.

13. The method of claim 12, wherein said composition comprises a detergent, chaeotropic agent or salt.

14. The method of claim 13, wherein said chaeotropic agent is urea.

15. The method of claim 1, further comprising collecting said protein or peptide molecules.

16. The method of claim 1, wherein said cellular source is a cell selected from the group consisting of a bacterial cell, a yeast cell, a fungal cell, an animal cell, a cell infected by a virus and a plant cell.

17. The method of claim 16, wherein said bacterial cell is an Escherichia coli cell.

18. The method of claim 16, wherein said yeast cell is a Sacchromyces cell.

19. An isolated protein or peptide molecule produced by the method of claim 1.

20. A composition for use in isolating a protein or peptide molecule or a population of protein or peptide molecules, said composition comprising:
(a) one or more cellular sources of said protein or peptide molecules;
(b) one or more pore-containing matrices which substantially retard the flow of high molecular weight molecules, structures, and aggregates but do not substantially retard the flow of soluble protein and peptide molecules; and optionally
(c) at least one compound or composition that lyses/disrupts/permeabilizes said cellular source.

21. An apparatus for extracting and isolating protein or peptide molecules, comprising:
(a) a housing; and
(b) one or more pore-containing matrices, which substantially retard the flow of high molecular weight molecules, structures, and aggregates but do not substantially retard the flow of said protein and peptide molecules in said container; and
(c) at least one composition selected from the group consisting of chromatographic resins that bind proteins or peptides, chromatographic resins that bind impurities, chromatographic resins having bound thereto protein modifying reagents, chromatographic resins having bound thereto enzymes, chromatographic resins having bound thereto nucleic acids, chromatographic resins having bound thereto an enzyme substrate, filters, and compositions capable of being used for detecting or quantifying the amount of protein or nucleic acid present in the sample.

22. The apparatus of claim 21, further comprising a porous solid support.

23. The apparatus of claim 21, wherein said pore containing matrix divides said tube into a sample application section and a sample collection section.

24. The apparatus of claim 21, wherein said pore containing matrix is selected from the group consisting of: a frit, a plug, a cartridge, or a swab tip.

25. The apparatus of claim 21, wherein said pore containing matrix is selected from the group consisting of: polyester, polyolefin, scintered polyethylene, nitrocellulose, cellulose acetate, nylon, cellulose, porous ceramic, silica, polysaccharide, and polymer.

26. The apparatus of claim 21, wherein said pore containing matrix is a solid matrix.

27. The apparatus of claim 21, wherein said pore containing matrix is a semi solid matrix.

28. The apparatus of claim 21, wherein the average size of said pores in said matrix range from about 0.1 to about 10,000 microns in diameter.

29. The apparatus of claim 23, wherein said sample collection section has an access port formed therein.

30. The apparatus of claim 21, wherein said pore containing matrix comprises a cell lysis/disruption/permeabilization composition.

31. The apparatus of claim 30, wherein said cell lysis/disruption/permeabilization composition is selected from the group consisting of a detergent, an enzyme, an inorganic salt, an acid, a base, and a buffering agent.

32. The apparatus of claim 21, wherein said housing is a tube, a bottle, a vial, an ampule, a microspin tube, a well, a column, a mini-column, a multi-well plate, a bag, a box, or a carton.

33. A kit for use in isolating a protein or peptide molecule or a population of protein or peptide molecules, said kit comprising the apparatus of claim 21.

34. The kit of claim 33, further comprising at least one composition selected from the group consisting of chromatographic resins that bind proteins or peptides, chromatographic resins that bind impurities, chromatographic resins having bound thereto protein modifying reagents, chromatographic resins having bound thereto enzymes, chromatographic resins having bound thereto nucleic acids, chromatographic resins having bound thereto an enzyme substrate, filters, and compositions capable of being used for detecting or quantifying the amount of protein or nucleic acid present in the sample.

35. The kit of claim 33, further comprising at least one composition selected from the group consisting of antibodies which bind to the protein or peptides of the invention, substrates for said protein or peptides, ligands for said proteins or peptides, cofactors for said protein or peptides, nucleic acid molecules which bind to said proteins or peptides, inhibitors of said proteins or peptides, enzymes which modify said proteins or peptides, compositions which modify said proteins or peptides, compositions which bind said proteins or peptides, compositions which are bound by said proteins or peptides, and compositions capable of being used for detecting or quantifying the amount of protein or nucleic acid present in the sample.

36. The kit of claim 33, wherein all compositions are contained in one or more fluid channels so that the sample may pass through the at least one pore-containing matrix and directly contact the at least one composition of claim 34 or 35 without the need for removal and re-application of the sample.

37. The kit of claim 33, wherein all compositions are contained in two or more fluid channels or containers such that the sample may be directly applied to the at least one composition of claim 34 or 35, after passing through the at least one pore-containing matrix.
